(19) United States
(12) Patent Application Publication
(10) Pub. No.: US 2016/0097712 A1
(43) Pub. Date: Apr. 7, 2016

(54) SIGNAL DETECTION METHOD, CALIBRATION CURVE CREATION METHOD, QUANTIFICATION METHOD, SIGNAL DETECTION DEVICE, MEASURING DEVICE, AND GLUCOSE CONCENTRATION MEASURING DEVICE

(71) Applicant: Seiko Epson Corporation, Tokyo (JP)

(72) Inventors: Sakiko SHIMIZU, Matsumoto (JP); Kazuhiro NISHIDA, Matsumoto (JP)

(21) Appl. No.: 14/873,513

(22) Filed: Oct. 2, 2015

(30) Foreign Application Priority Data
Oct. 7, 2014 (JP) 2014-206460
Oct. 15, 2014 (JP) 2014-210486
May 14, 2015 (JP) 2015-098825

(51) Int. Cl. G01N 21/27; G01N 33/49; G01N 21/31

(52) U.S. Cl. CPC G01N 21/274; G01N 21/31; G01N 33/49; G01N 2201/12746

(57) ABSTRACT

A signal detection method includes acquiring a measurement signal including a first signal, which is a signal of a target component, and a second signal, which is a signal of an interference component; and performing an orthogonal operation for adjusting the measurement signal such that the measurement signal is orthogonal to the second signal.

### BACKGROUND

1. Technical Field

The present disclosure relates to a signal detection method, a calibration curve creation method, a quantification method, a signal detection device, a measuring device, and a glucose concentration measuring device.

2. Related Art

Various techniques for analyzing a signal of a predetermined component included in a measurement signal are known. One such technique is an independent component analysis.

For example, JP-A-2007-44104 discloses a technique of analyzing the concentration of a target component included in an observation signal, which is a measurement signal from a body, by performing independent component analysis of the observation signal. JP-A-2007-44104 expresses the observation signal as a linear sum of a basic function with the calculated independent component as the basic function.

In addition, JP-A-2013-36973 discloses a technique of performing independent component analysis of observation data that is a measurement signal from a body, calculating a mixing coefficient for a target component included in the observation data, and acquiring a calibration curve from the mixing coefficient and the content of the target component of original observation data.

A signal relevant to the independent component is preferably a signal of a unique component. Accordingly, since there is no influence of other components, the signal relevant to the independent component is “independent” of the other components. In practice, however, each independent component extracted from mixed components by the independent component analysis may not be completely “independent”. In such a case, even if the independent component analysis is performed to detect the concentration of 1% or less of a trace component in a measurement target, it can be difficult to accurately detect the concentration of the trace component.

### SUMMARY

The present disclosure proposes a technique capable of accurately detecting a signal relevant to a trace component included in a measurement signal such as a signal from a body.

### Application Example 1

A signal detection method according to this application example includes acquiring a measurement signal, where the measurement signal includes a first signal and a second signal different from the first signal; and performing an orthogonal operation for adjusting the measurement signal such that the measurement signal is orthogonal to the second signal.

According to a study by the inventors, it has been found that a vector representing the first signal is orthogonal to a vector representing the second signal, and the first and second signals form an orthogonal vector space. Therefore, in the signal detection method according to this application example, an orthogonal operation for acquiring a signal corresponding to the first signal by making the measurement signal orthogonal to the second signal. Therefore, by removing the second signal from the measurement signal, it is possible to detect the first signal with improved accuracy. As a result, it is possible to accurately detect the concentration of the component relevant to the first signal in the sample containing the component relevant to the first signal and the component relevant to the second signal.

### Application Example 2

In the signal detection method according to the application example, it is preferable that a second feature signal (i.e., second sample feature signal) obtained by performing multivariate analysis processing of a second sample signal is used in the orthogonal operation. The second sample signal is obtained by measuring a sample that contains a component relevant to the second signal and does not contain a component relevant to the first signal.

In the signal detection method according to this application example, the second feature signal that is the feature quantity of the component relevant to the second signal can be extracted by performing multivariate analysis processing of the second sample signal obtained by measuring the sample that contains the component relevant to the second signal and does not contain the component relevant to the first signal. In addition, since the orthogonal operation for making the measurement signal orthogonal to the acquired second feature signal is performed, it is possible to effectively remove the second signal from the measurement signal obtained by measuring the sample containing the component relevant to the first signal and the component relevant to the second signal.

### Application Example 3

In the signal detection method according to the application example, it is preferable that the multivariate analysis processing is an independent component analysis.

In the signal detection method according to this application example, the independent component analysis process is used as the multivariate analysis processing on the second sample signal. Accordingly, in particular, when the component relevant to the second signal is a high percentage component, it is possible to detect the second feature signals (second sample feature signals) that are strongly orthogonal to each other and that have little error.

### Application Example 4

In the signal detection method according to the application example, the orthogonal operation may be a projection operation for projecting the measurement signal to a space orthogonal to a space extended by the second feature signal (second sample feature signal).

In the signal detection method according to this application example, the second signal is removed from the measurement signal including the first and second signals by performing a projection operation for projecting the measurement signal to the space orthogonal to the space extended by the second feature signal (second sample feature signal). Therefore, it is possible to detect the first signal with high accuracy.

### Application Example 5

In the signal detection method according to the application example, with the measurement signal provided as a measurement vector M, the first signal provided as a first vector M₀, the second feature signal (second sample feature signal) provided as γ interference unit vectors Pₖ, the space extended by the second feature signal (second sample feature signal) provided as a matrix P including the interference unit vectors Pₖ, a pseudo-inverse matrix of the matrix P is expressed as P⁺, and a unit matrix provided as E, the projection operation is expressed by Equation (1).

\vec{M₀}=(E−P·P⁺)\vec{M} (1)

In the signal detection method according to this application example, the first signal (the first vector M₀) included in the measurement signal expressed as the measurement vector M can be detected with high accuracy by performing the projection operation expressed as Equation (1).

### Application Example 6

In the signal detection method according to the application example, in the orthogonal operation, an orthogonalization method of Gram-Schmidt using the second feature signal (second sample feature signal) may be applied for the measurement signal.

In the signal detection method according to this application example, the second signal (second sample feature signal) is removed from the measurement signal including the first and second signals by applying the orthogonalization method of Gram-Schmidt using the second feature signal (second sample feature signal) for the measurement signal. Therefore, it is possible to detect the first signal with high accuracy.

### Application Example 7

In the signal detection method according to the application example, with the measurement signal provided as a measurement vector M, the first signal provided as a first vector M₀, the second feature signal (second sample feature signal) provided as γ interference unit vectors Pₖ, γ intermediate vectors provided as Wₖ, and transposed vectors of the intermediate vectors Wₖ provided as Wₖ^{T}, the orthogonalization method of Gram-Schmidt is expressed by Equations (2) and (3) with a first intermediate vector W₁ as a first interference unit vector P₁.

$$\begin{matrix}\vec{{W}_{t}} = \vec{{P}_{t}} - {\sum }_{i = 1}^{t - 1} \frac{{\vec{{W}_{i}}}^{T} \cdot \vec{{P}_{t}}}{{\vec{{W}_{i}}}^{T} \cdot \vec{{W}_{i}}} \cdot \vec{{W}_{i}} , t = 2 … γ & ( 2 ) \\ \vec{{M}_{0}} = \vec{M} - {\sum }_{i = 1}^{γ} \frac{{\vec{{W}_{i}}}^{T} \cdot \vec{M}}{{\vec{{W}_{i}}}^{T} \cdot \vec{{W}_{i}}} \cdot \vec{{W}_{i}} & ( 3 )\end{matrix}$$

In the signal detection method according to this application example, the γ intermediate vectors Wₖ are sequentially orthogonalized by the orthogonalization method of Gram-Schmidt expressed as Equations (2) and (3). Accordingly, the measurement vector M is orthogonal to each of the γ intermediate vectors Wₖ. As a result, the measurement vector M is orthogonal to all of the second signals. Thus, the first signal (first vector M₀) included in the measurement signal expressed as the measurement vector M can be detected with high accuracy.

### Application Example 8

In the signal detection method according to the application example, a percentage of the first signal in the measurement signal may be equal to or less than 1%.

In the signal detection method according to this application example, the amount of the component relevant to the first signal is small, and the first signal of the trace component in the measurement signal can be detected with high accuracy even when the first signal is included in the measurement signal at the percentage of 1% or less.

### Application Example 9

A calibration curve creation method according to this application example includes: calculating an inner product value between the first signal, which is obtained by executing the signal detection method according to any one of application examples, and a unit signal of the first signal; and creating a calibration curve showing a relationship between a physical quantity relevant to the first signal and the inner product value.

In the calibration curve creation method according to this application example, the calibration curve is created by calculating the inner product value between the first signal, which is obtained by executing the signal detection method capable of detecting the first signal from the measurement signal with high accuracy, and the unit signal of the first signal. Therefore, it is possible to create a high-accuracy calibration curve.

### Application Example 10

A quantification method according to this application example includes calculating an inner product value between the first signal, which is obtained by executing the signal detection method according to any one of application examples, and a unit signal of the first signal.

In the quantification method according to this application example, since the inner product value between the first signal, which is obtained by executing the signal detection method capable of detecting the first signal from the measurement signal with high accuracy, and the unit signal of the first signal is taken, it is possible to calculate the magnitude (scalar quantity) of the first signal in the vector space with high accuracy.

### Application Example 11

In the quantification method according to the application example, the method may further include quantifying a physical quantity with reference to the inner product value and a calibration curve.

In the quantification method according to this application example, since the inner product value between the first signal and the unit signal of the first signal and the calibration curve showing the relationship between the inner product value and the physical quantity relevant to the first signal are referred to, it is possible to correctly quantify the physical quantity of the component relevant to the first signal in the sample containing the component relevant to the first signal and the component relevant to the second signal.

### Application Example 12

In the quantification method according to the application example, it is preferable that the calibration curve is obtained by the calibration curve creation method according to the application example.

In the quantification method according to this application example, since the calibration curve showing the relationship between the inner product value and the physical quantity relevant to the first signal is used, it is possible to quantify the physical quantity of the component relevant to the first signal contained in the measurement target with high accuracy.

### Application Example 13

In the quantification method according to the application example, the physical quantity may be glucose concentration in blood.

In the quantification method according to this application example, it is possible to quantify the physical quantity of glucose (component relevant to the first signal) contained in a small amount with respect to water (component relevant to the second signal) contained at a high percentage in blood with high accuracy.

### Application Example 14

A signal detection device according to this application example includes: an acquisition unit that acquires a measurement signal by measuring a measurement target containing a component relevant to the first signal and a component relevant to the second signal different from the first signal; and an arithmetic processing unit that performs an orthogonal operation for making the measurement signal orthogonal to the second signal.

According to the configuration according to this application example, the acquisition unit acquires a measurement signal by measuring the measurement target containing the component relevant to the first signal and the component relevant to the second signal. In addition, the arithmetic processing unit forms a vector space where the vector representing the second signal is orthogonal to the vector representing the first signal, and performs an orthogonal operation for making the measurement signal orthogonal to the second signal in the vector space. Therefore, it is possible to realize a signal detection device capable of detecting the first signal with high accuracy by removing the second signal from the measurement signal including the first and second signals.

### Application Example 15

A measuring device according to this application example includes: an acquisition unit that acquires a measurement signal by measuring a measurement target containing a component relevant to the first signal and a component relevant to the second signal different from the first signal; and an arithmetic processing unit that performs an orthogonal operation for making the measurement signal orthogonal to the second signal and quantifies a physical quantity using a result of the orthogonal operation.

According to the configuration according to this application example, the acquisition unit acquires a measurement signal by measuring the measurement target containing the component relevant to the first signal and the component relevant to the second signal. In addition, the arithmetic processing unit forms a vector space where the vector representing the second signal is orthogonal to the vector representing the first signal, performs an orthogonal operation for making the measurement signal orthogonal to the second signal in the vector space, and quantifies the physical quantity using the operation result. Therefore, it is possible to realize a measuring device capable of detecting the first signal by removing the second signal from the measurement signal including the first and second signals and quantifying the physical quantity of the component relevant to the first signal with high accuracy.

A first aspect of the present disclosure is directed to a signal detection method including: acquiring a measurement signal (signal from the body) by measuring a predetermined measurement target, the measurement signal including a second signal (interference signal) that is a signal of a high percentage component and a first signal (target signal) that is a signal of a trace component; and performing an orthogonal operation for making the measurement signal (signal from the body) orthogonal to the second signal (interference signal) in a vector space where vectors representing the signals of the respective components are orthogonal to each other.

According to the first aspect of the present disclosure, it is possible to obtain a signal excluding a high percentage component by performing an orthogonal operation for making the measurement signal (signal from the body) orthogonal to the second signal (interference signal), which is a signal of a high percentage component, in the vector space where the vectors representing the signals of the respective components are orthogonal to each other. Since the signal of the high percentage component is removed, it is possible to detect the first signal (target signal), which is a trace component in the measurement signal (signal from the body), with high accuracy.

A second aspect of the present disclosure is directed to the signal detection method according to the first aspect of the present disclosure, in which a percentage of the first signal (target signal) in the measurement signal (signal from the body) is equal to or less than 1%.

According to the second aspect of the present disclosure, even if the first signal (target signal) is slightly included in the measurement signal (signal from the body) at the percentage of 1% or less, it is possible to achieve the same effect as in the first aspect of the present disclosure.

A third aspect of the present disclosure is directed to the signal detection method according to the first or second aspect of the present disclosure, in which a percentage of the second signal (interference signal) in the measurement signal (signal from the body) is equal to or greater than 3%.

A fourth aspect of the present disclosure is directed to the signal detection method according to any one of the first to third aspects of the present disclosure, in which the orthogonal operation is performed by using a signal obtained by performing independent component analysis of the second sample signal (signal of only an interference component) that is obtained by measuring a predetermined sample that contains a component relevant to the second signal (interference signal) and does not contain a component relevant to the first signal (target signal).

According to the fourth aspect of the present disclosure, it is possible to perform the orthogonal operation using the signal obtained by performing multivariate analysis of the second sample signal (signal of only an interference component) that is obtained by measuring a predetermined sample that contains a component relevant to the second signal (interference signal) and does not contain a component relevant to the first signal (target signal). Therefore, it is possible to effectively remove the component relevant to the second signal (interference signal). As the multivariate analysis, it is possible to use various analysis methods, such as an independent component analysis or a main component analysis. Among these, it is most preferable to use the independent component analysis of the strongest independence as the multivariate analysis since it is possible to detect a signal relevant to the trace component with high accuracy.

Specifically, for example, the performing of the orthogonal operation may be configured to include performing a projection operation for projecting the measurement signal (signal from the body) to a predetermined orthogonal subspace that is orthogonal to the second signal (interference signal), as a fifth aspect of the present disclosure.

The performing of the orthogonal operation may be configured to include making the measurement signal (signal from the body) orthogonal to the second signal (interference signal) using an orthogonalization method of Gram-Schmidt, as a sixth aspect of the present disclosure.

A seventh aspect of the present disclosure is directed to the signal detection method according to any one of the first to sixth aspects of the present disclosure, in which the high percentage component of the measurement target is water, and the acquisition of the measurement signal (signal from the body) includes acquiring the measurement signal (signal from the body) as spectrum data.

According to the seventh aspect of the present disclosure, it is possible to acquire the measurement signal (signal from the body) of the measurement target, of which a high percentage component is water, as spectrum data.

An eighth aspect of the present disclosure is directed to the signal detection method according to the seventh aspect of the present disclosure, in which the acquisition of the spectrum data includes acquiring spectrum data of the measurement target at different temperatures.

According to the eighth aspect of the present disclosure, for example, there is a temperature characteristic in the spectrum data (or the composition ratio of feature quantities) of water. Therefore, it is possible to detect the first signal (target signal) in consideration of the temperature characteristic.

A ninth aspect of the present disclosure is directed to a calibration curve creation method including: executing the signal detection method according to any one of the first to eighth aspects of the present disclosure for a plurality of the measurement targets having different component concentrations relevant to the first signal (target signal); and creating a calibration curve for the component concentration relevant to the first signal (target signal).

According to the ninth aspect of the present disclosure, it is possible to create the calibration curve of the component concentration relevant to the first signal (target signal) included in the measurement target.

A tenth aspect of the present disclosure is directed to a concentration measuring method including: executing the signal detection method according to any one of the first to seventh aspects of the present disclosure for the measurement target whose component concentration relevant to the first signal (target signal) is unknown; and measuring the unknown component concentration using the detected signal and the calibration curve created by executing the calibration curve creation method according to the ninth aspect of the present disclosure.

According to the tenth aspect of the present disclosure, the component concentration relevant to the first signal (target signal) included in the measurement target can be accurately calculated by using the calibration curve created according to the ninth aspect of the present disclosure.

An eleventh aspect of the present disclosure is directed to a signal detection device including: an acquisition unit that acquires a measurement signal (signal from the body) by measuring a predetermined measurement target, the measurement signal including a second signal (interference signal) that is a signal of a high percentage component and a first signal (target signal) that is a signal of a trace component; and an arithmetic processing unit that performs an orthogonal operation for making the measurement signal (signal from the body) orthogonal to the second signal (interference signal) in a vector space where vectors representing the signals of the respective components are orthogonal to each other.

According to the eleventh aspect of the present disclosure, it is possible to realize a signal detection device that exhibits the same effect as in the first aspect of the present disclosure.

### BRIEF DESCRIPTION OF THE DRAWINGS

The present disclosure will be described with reference to the accompanying drawings, wherein like numbers reference like elements.

FIG. 1 is a diagram for explaining the concept of the present embodiment.

FIG. 2 is a block diagram illustrating the configuration of a signal detection device according to the present embodiment.

FIG. 3 is a flowchart showing the flow of the interference component feature quantity extraction process according to the first embodiment.

FIGS. 4A and 4B are diagrams showing the data obtained by the interference component feature quantity extraction processing according to the first embodiment.

FIG. 5 is a flowchart showing the flow of the calibration curve creation process according to the first embodiment.

FIGS. 6A and 6B are diagrams showing the data obtained by the calibration curve creation process according to the first embodiment.

FIG. 7 is a diagram showing an example of the calibration curve created by the calibration curve creation process according to the first embodiment.

FIG. 8 is a diagram for explaining the orthogonalization reference vector obtained by the projection operation according to the first embodiment.

FIG. 9 is a flowchart showing the flow of the concentration measurement processing according to the first embodiment.

FIGS. 10A and 10B are diagrams showing the data obtained by the calibration curve creation process according to a second embodiment.

FIG. 11 is a block diagram illustrating the configuration of a measuring device according to a modification example 1.

FIGS. 12A and 12B are diagrams showing the comparison data when performing independent component analysis.

### DESCRIPTION OF EXEMPLARY EMBODIMENTS

Hereinafter, example embodiments will be described with reference to the accompanying diagrams.

### Principle

A physical quantity to be measured is a vector that is expressed as a linear sum of various physical quantities. That is, two or more physical quantity components are included in a measurement signal, where the measurement signal is a signal from a body obtained by measuring a measurement target. The measurement signal is expressed as a linear sum of the signals of the respective physical quantity components. The measurement signal is expressed as a linear sum of a target signal that is a first signal and an interference signal that is a second signal, and the first signal is orthogonal to the second signal.

According to a study conducted by the inventors, since the first and second signals are originally independent of each other, the first signal may be obtained by adjusting the measurement signal such that the measurement signal is orthogonal to the second signal. Therefore, by making the measurement signal orthogonal to the second signal, a signal corresponding to the first signal can be extracted with high accuracy.

An electrical signal, an audio signal, an electromagnetic wave signal, and the like can be considered as measurement targets of this application. The present disclosure can be applied to a case of measuring a specific signal component included in these signals or to a case of measuring the concentration or mass of a specific component contained in a measurement target, such as a gas or liquid. In the following embodiment, concentration is used as an example of the physical quantity component that is a measurement target. However, in the following embodiment, the physical quantity component is not limited to the concentration, and may be all kinds of variation parameters (concentration, temperature, pressure, and the like).

In addition, it is thought that the measurement signal is expressed as a linear sum of the signals of physical quantity components. Therefore, if the signal of each physical quantity component is expressed as a vector, it is possible to define the vector space where a vector representing a physical quantity component of an interference material (vector representing a second signal) is orthogonal to a vector representing a target physical quantity component (vector representing a first signal). As a result, the measurement signal vector can be defined in this vector space. In addition, the number of dimensions of the vector space is the number of independent physical quantity components included in the measurement signal.

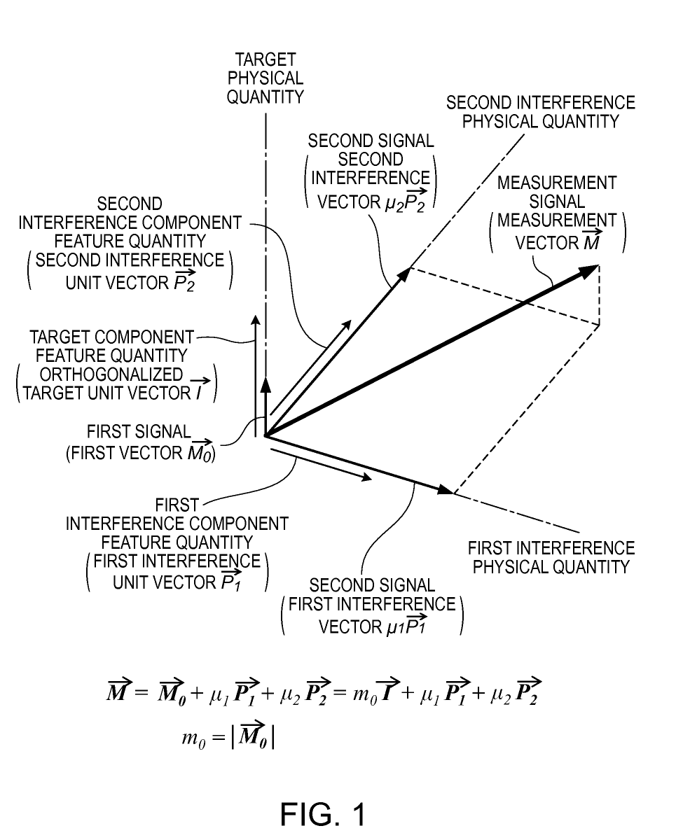

FIG. 1 is a diagram for explaining the concept of the present embodiment. Simplified vector space, a vector representing a measurement signal (referred to as a measurement vector M), and the like are drawn in FIG. 1. In the example shown in FIG. 1, the measurement vector M obtained from the measurement target is expressed as a linear sum of a first signal, which is one trace component and is a target signal, and a second signal, which are two high percentage components and interference components. The first signal is a signal representing the target physical quantity that is included in the measurement signal, and is expressed as a first vector M₀ in the example shown in FIG. 1. On the other hand, the second signal is a signal representing interference signals of the measurement signal, and is expressed as a vector sum of a first interference vector μ₁P₁ and a second interference vector μ₂P₂ in the example shown in FIG. 1.

The first signal (first vector M₀) is orthogonal to the second signal (vector sum of the first interference vector μ₁P₁ and the second interference vector μ₂P₂). Thus, the measurement vector M representing the measurement signal is expressed as a linear sum of the first signal (first vector M₀) representing the target physical quantity and the second signal (vector sum of all interference vectors, such as the first interference vector μ₁P₁ or the second interference vector μ₂P₂) representing the interference physical quantity, and the first and second signals are orthogonal to each other.

In FIG. 1, since the number of independent components included in the measurement signal is three, a vector space in which the measurement vector M is defined is expressed as a three-dimensional space. Specifically, in the example shown in FIG. 1, the linear sum of the first interference vector μ₁P₁ representing the first interference component that is the first high percentage component, the second interference vector μ₂P₂ representing the second interference component that is the second high percentage component, and the first vector M₀ representing a target component that is a trace component represents the measurement vector M.

In the example shown in FIG. 1, the first interference vector μ₁P₁ and the second interference vector μ₂P₂ are drawn orthogonal to each other. However, the respective interference vectors do not need to be orthogonal to each other. The first signal (first vector M₀) may be orthogonal to all interference vectors (in the example shown in FIG. 1, a vector sum of the first interference vector μ₁P₁ and the second interference vector μ₂P₂) that are the second signals.

For example, even if the first interference vector μ₁P₁ and the second interference vector μ₂P₂ form an oblique coordinate system, the first signal (the first vector M₀) may be orthogonal to the space where all interference vectors extend (in the example shown in FIG. 1, a plane defined by the first interference vector μ₁P₁ and the second interference vector μ₂P₂). A vector orthogonal to the space where all interference vectors extend is assumed to be the first vector M₀.

In the following embodiment, the trace component is a “target component”, and it is an object of the embodiment to correctly detect a signal relevant to the trace component from the measurement signal (signal from the body). Accordingly, it is an object of the embodiment to detect the first vector M₀ of the trace component from the measurement vector M representing a measurement signal (signal from the body). On the other hand, the high percentage component can be said to be a component that inhibits the detection of a signal relevant to the trace component from the measurement signal (signal from the body), the high percentage component is referred to as an “interference component”.

Meanwhile, as a method of signal processing for analyzing how much each component is contained, an independent component analysis is known. When measuring the amount (or may be a percentage or concentration) of a specific component contained in the measurement target using the independent component analysis, a problem may occur. Specifically, when a specific component contained in the measurement target is a trace component whose percentage is extremely small compared with the percentage of other components, the independent component analysis has a problem that it is difficult to correctly determine the content (or may be a percentage or concentration) of the trace component.

The independent component analysis is a technique of estimating the number and amount of contained components based on a statistical method using a random variable. Therefore, when one independent component included in the measurement signal (signal from the body) is a trace component whose percentage is 1% or less, it may be difficult to measure the trace component correctly.

FIG. 1 is a diagram for explaining the principle of the present disclosure, and the problem of quantification by the independent component analysis that the present inventors have found will be described with reference to FIG. 1. When one trace component is completely independent of two high percentage components, the first vector M₀ of the trace component is orthogonal to the vector sum of the first interference vector μ₁P₁ and the second interference vector μ₂P₂. That is, it is possible to correctly measure the amount of each target component regardless of whether the amount is large or small.

However, in the practical independent component analysis, a trace of one target component cannot be completely independently separated from the interference components (two high percentage components). Therefore, the present inventors have found that a state in which the target component includes a slight error of interference components is typically regarded as “independent”. This is because the independent component analysis is an analysis based on the statistical method using a random variable.

It does not matter that the target component cannot be separated completely independent of the interference components in the independent component analysis since the degree of separation can be expressed as the degree of orthogonality between the first signal that is a target component and the second signal that is an interference component. That is, the target signal obtained directly by the independent component analysis is shifted from the normal of the plane defined by the first interference vector μ₁P₁ and the second interference vector μ₂P₂.

Even if the shift of the degree of orthogonality of the target signal (inclination of the target signal with respect to the normal of interference components) is a slight error of approximately 1/100, the influence of the interference components cannot be neglected since the amount of the target component is very small. If the first signal that is a target component is completely orthogonal to the second signal that is an interference component, the amount of the target component can be accurately measured regardless of whether the amount is large or small.

However, since the target signal obtained directly by the independent component analysis is not completely orthogonal to the interference components, the amount of high percentage components that are slightly contained affects the amount of the trace component. On the other hand, as described herein, since a component of the measurement signal orthogonal to the interference components is considered to be the first signal, it is natural that the influence of the interference components can be significantly reduced compared with that in the related art.

After all, for conventional quantification based on independent component analysis, even if there is only a slight error in the content of the extracted high percentage component, the error affects the content of a trace component. This causes a large change for the trace component. Therefore, it can be said that, a method for determining the amount (or may be a percentage or concentration) of a trace component, the quantification based on only independent component analysis is not suitable for detecting a small amount of the trace component.

The high percentage component is a component whose amount (or may be a percentage or concentration) can be determined with high accuracy by the independent component analysis, and the percentage of the high percentage component in the measurement signal is 3% or more, for example.

In order to solve the above problem, in the present embodiment, the signal of the target component that is a trace component is detected using orthogonalization (in the present embodiment, also referred to as “orthogonal operation”) that is a method of signal processing. Specifically, the first signal (first vector M₀) of the target component that is a trace component is detected by making the measurement vector M orthogonal to the vector (vector sum of the first interference vector μ₁P₁ and the second interference vector μ₂P₂) representing the second signal of the interference component that is a high percentage component.

Since the second signal of the interference component is a signal of a high percentage component that is sufficiently included in the measurement signal (signal from the body), the independent component analysis is effective. Therefore, by preparing a sample of the measurement target and analyzing the measurement signal of the sample, which contains an interference component and does not contain a target component, through the independent component analysis, it is possible to calculate the interference component feature quantity (for example, a first interference unit vector P₁ or a second interference unit vector P₂ shown in FIG. 1) that is an independent component of the interference component contained in the sample.

### Signal Detection Device

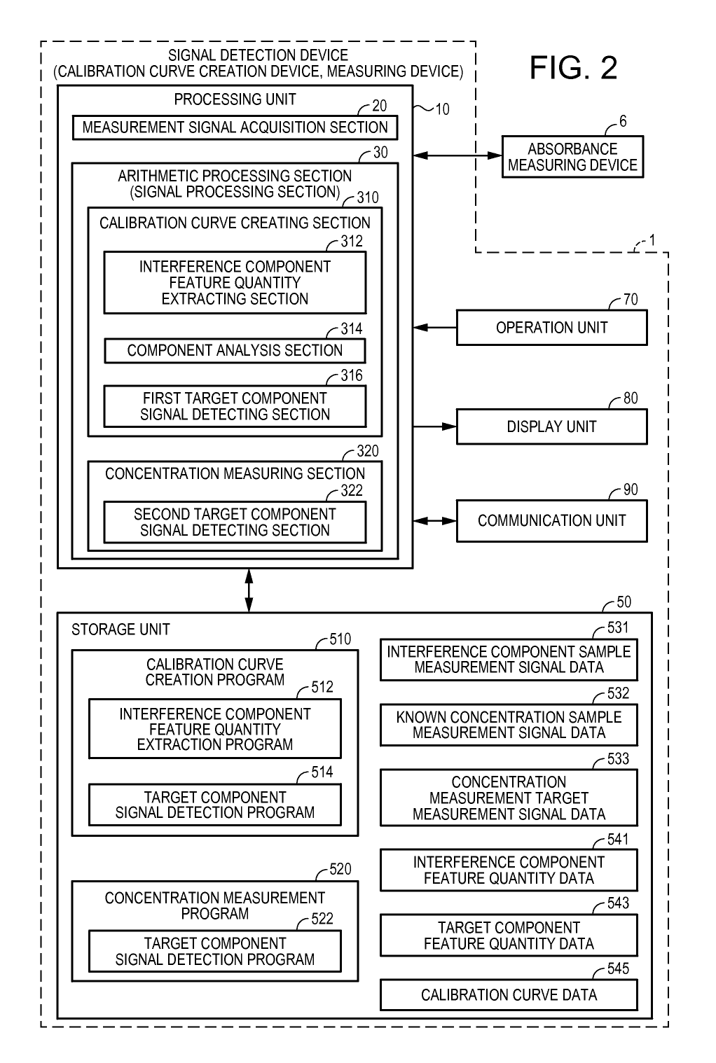

Next, an example of the configuration of a signal detection device to which the present disclosure is applied will be described. FIG. 2 is a block diagram illustrating the configuration of a signal detection device according to the present embodiment. Since a signal detection device 1 according to the present embodiment has functions of a signal detection device, a calibration curve creation device, and a measuring device, the signal detection device 1 can also be referred to as a calibration curve creation device or a measuring device. Although the signal detection device 1 will be described as being configured separately from an absorbance measuring device 6, the signal detection device 1 may also be configured to include the absorbance measuring device 6.

The signal detection device 1 is a kind of electronic computer system including a processing unit 10, a storage unit 50, an operation unit 70, a display unit 80, and a communication unit 90. The processing unit 10 is realized, for example, by a microprocessor, such as a central processing unit (CPU) or a graphics processor unit (GPU), or an electronic component, such as an application specific integrated circuit (ASIC), a field-programmable gate array (FPGA), or an integrated circuit (IC) memory. In addition, the processing unit 10 controls input and output of data to and from each functional unit, and calculates the concentration of the target component contained in the measurement target by performing various kinds of arithmetic processing based on a predetermined program or data, an operation input signal from the operation unit 70, measurement results of the absorbance measuring device 6, and the like.

The processing unit 10 includes a measurement signal acquisition section 20 as an acquisition section and an arithmetic processing section 30. The measurement signal acquisition section 20 controls the absorbance measuring device 6 by performing predetermined communication with the absorbance measuring device 6, and acquires the result measured by the absorbance measuring device 6 as a measurement signal. The measurement signal may be an analog signal. In this case, however, it is assumed that the measurement signal is converted into measurement signal data, which is a digital signal, by the measurement signal acquisition section 20. The absorbance measuring device 6 is a device for measuring the absorbance spectrum showing the absorbance for the wavelength of each light beam by emitting various light beams having different wavelengths to the measurement target and receiving the transmitted light that has been transmitted through the measurement target. That is, the measurement signal is expressed as an absorbance spectrum.

There are three measurement targets of the absorbance measuring device 6. These are an interference component sample that is a sample of an interference component that does not contain a target component, a known concentration sample that is a sample containing a target component whose concentration is known or is determined by separate measurement, and a concentration measurement target containing a target component whose concentration is unknown and is to be measured. The measured absorbance spectrum is stored in the storage unit 50, as interference component sample measurement signal data 531, known concentration sample measurement signal data 532, and concentration measurement target measurement signal data 533, by the measurement signal acquisition section 20.

The arithmetic processing section (signal processing section) 30 is a processing section that performs various kinds of digital signal processing on the measurement signal acquired by the measurement signal acquisition section 20, and can be said to be a kind of signal processing section. The arithmetic processing section 30 includes a calibration curve creating section 310 and a concentration measuring section 320.

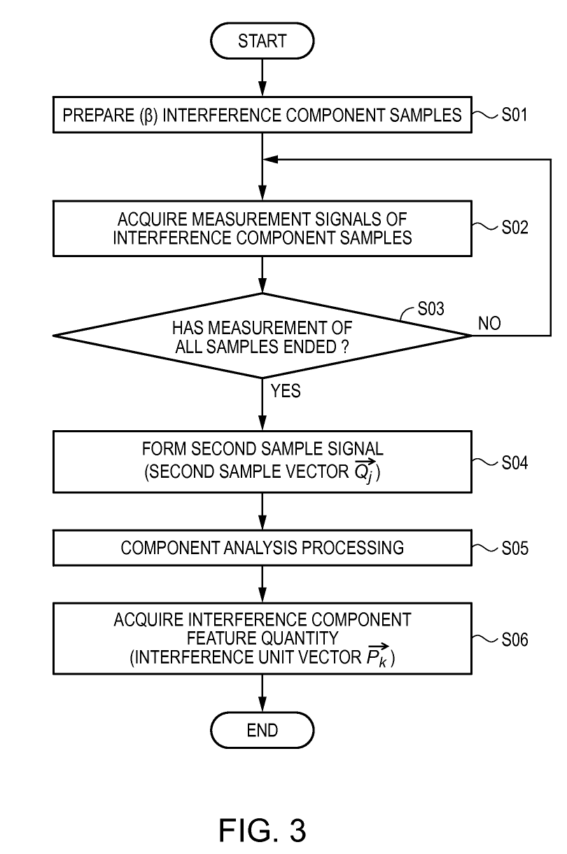

The calibration curve creating section 310 performs a calibration curve creation process (refer to FIG. 3) according to a calibration curve creation program 510 stored in the storage unit 50, and creates a calibration curve for calculating the concentration of a target component contained in the concentration measurement target. The calibration curve creating section 310 includes an interference component feature quantity extracting section 312, a component analysis section 314, and a first target component signal detecting section 316.

The interference component feature quantity extracting section 312 performs an interference component feature quantity extraction process according to an interference component feature quantity extraction program 512 that is a subroutine program of the calibration curve creation program 510. The component analysis section 314 performs component analysis processing (multivariate analysis processing) of interference components of the measurement signal. The first target component signal detecting section 316 performs a target component signal detection process for detecting the signal of the target component from a sample having a known concentration according to a target component signal detection program 514 that is a subroutine program of the calibration curve creation program 510.

The concentration measuring section 320 performs a concentration measurement process according to a concentration measurement program 520. Specifically, the concentration measuring section 320 measures the concentration of the target component contained in the concentration measurement target using the calibration curve created by the calibration curve creating section 310. The concentration measuring section 320 includes a second target component signal detecting section 322. The second target component signal detecting section 322 performs a target component signal detection process for detecting the signal of the target component contained in the concentration measurement target, that is, the first signal (first vector M₀) according to a target component signal detection program 522 that is a subroutine program of the concentration measurement program 520.

The measurement signal acquisition section 20 and the arithmetic processing section 30 may also be formed by an electronic circuit that performs signal processing, rather than as a software-based functional section that is realized by executing a program as described above. Although the first target component signal detecting section 316 and the second target component signal detecting section 322 have been described as separate functional sections, the first target component signal detecting section 316 and the second target component signal detecting section 322 may be designed as a common functional section.

The storage unit 50 is realized by a storage medium, such as an IC memory, a hard disk, or an optical disc, and stores various programs or various kinds of data, such as data during the calculation process of the processing unit 10. The connection between the processing unit 10 and the storage unit 50 is not limited to a connection using an internal bus circuit in the device, and may be realized by using a communication line, such as a local area network (LAN) or the Internet. In this case, the storage unit 50 may be realized by using a separate external storage device from the signal detection device 1.

The calibration curve creation program 510 and the concentration measurement program 520 are stored in the storage unit 50. The calibration curve creation program 510 includes, as subroutine programs, the interference component feature quantity extraction program 512 for executing the interference component feature quantity extraction process and the target component signal detection program 514 for creating a calibration curve. The concentration measurement program 520 includes, as a subroutine program, the target component signal detection program 522 for measuring the concentration of the concentration measurement target.

In addition, the storage unit 50 stores the interference component sample measurement signal data 531, the known concentration sample measurement signal data 532, the concentration measurement target measurement signal data 533, an interference component feature quantity data 541, a target component feature quantity data 543, and a calibration curve data 545 that are calculated when performing the interference component feature quantity extraction process, the calibration curve creation process, and the concentration measurement process. In addition to these, the storage unit 50 can appropriately store temporary data that is calculated when performing each process.

The operation unit 70 receives various kinds of operation input performed by the user, and outputs an operation input signal corresponding to the operation input to the processing unit 10. For example, the operation unit 70 can be realized by a button switch, a lever switch, a dial switch, a track pad, a mouse, a keyboard, a touch panel, and the like.

The display unit 80 displays a calculation result of the processing unit 10, a guidance display showing the operation procedure, and the like. For example, the display unit 80 can be realized by a liquid crystal display, a touch panel, or the like.

The communication unit 90 realizes a communication function for data exchange between the signal detection device 1 and an external device by connecting the signal detection device 1 to the external device. The communication mode may be wired or may be wireless. In addition, the communication unit 90 may be connectable to the Internet circuit or to a public communication network.

Signal detection method, calibration curve creation method, and quantification method

### First Embodiment

Next, a signal detection method, a calibration curve creation method, and a quantification method according to the first embodiment will be described. The signal detection method, the calibration curve creation method, and the quantification method according to the first embodiment include an interference component feature quantity extraction process, a calibration curve creation process, and a concentration measurement process.

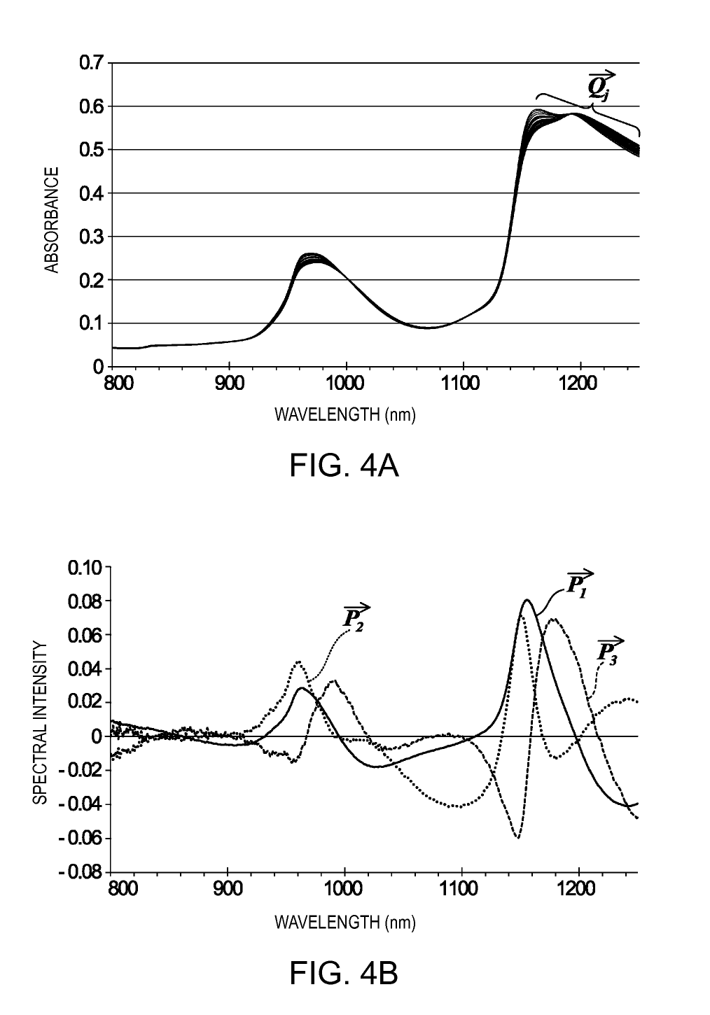

First, the interference component feature quantity extraction process according to the first embodiment will be described. FIG. 3 is a flowchart showing the flow of the interference component feature quantity extraction process according to the first embodiment. FIGS. 4A and 4B are diagrams showing the data obtained by the interference component feature quantity extraction process according to the first embodiment. Specifically, FIG. 4A is a diagram showing an example of the absorbance spectrum obtained from the interference component sample, and FIG. 4B is a diagram showing an example of the spectrum of the interference unit vector acquired by the independent component analysis process.

In the first embodiment, a method of acquiring the first signal will be described by way of an example in which the signal detection device 1 calculates the concentration of glucose contained in the aqueous glucose solution having an unknown concentration. The aqueous glucose solution of the measurement target contains glucose as a target component (target physical quantity) at a concentration of 1% or less, and contains water as an interference component (interference physical quantity) at a concentration of 90% or more that is equal to or greater than 3%. Therefore, glucose as a target component is a trace component, and water as an interference component is a high percentage component.

The interference component feature quantity extraction process is a process for extracting the feature quantity of the interference component from the measurement signal (second sample signal) of the interference component sample that contains an interference component relevant to the second signal and does not contain a target component relevant to the first signal. In the present embodiment, the interference component sample is a component other than glucose that is a target component, that is, water that is a high percentage component. The interference component feature quantity extraction process is realized by executing the interference component feature quantity extraction program 512 that is a subroutine program included in the calibration curve creation program 510 shown in FIG. 2.

In step S01 shown in FIG. 3, a plurality of interference component samples that contain an interference component relevant to the second signal and do not contain a target component relevant to the first signal are prepared. The spectrum data (or the composition ratio of feature quantities) of water that is an interference component sample changes with the temperature. Accordingly, a plurality (β; β is an integer of 2 or more) of water components obtained by changing the temperature are prepared as interference component samples.

In step S02, measurement signals (second sample signals) of β water components having different temperatures are acquired. Here, an absorbance spectrum is acquired as a measurement signal of water that is an interference component sample. The absorbance spectrum of the interference component sample is acquired from the absorbance measuring device 6 through the measurement signal acquisition section 20, and is stored in the storage unit 50 as the interference component sample measurement signal data 531. This is repeated until the measurement of β interference component samples ends (step S03: NO to step S02).

When the measurement for all (β) interference component samples is completed (step S03: YES), data of the absorbance spectrum of water that is an interference component is obtained from the interference component samples as a result. FIG. 4A shows the absorbance spectrum obtained from the interference component sample (water). In FIG. 4A, the horizontal axis indicates a measurement point (i: 1 to α, α is an integer of 2 or more) corresponding to the wavelength of light, and the vertical axis indicates the intensity of the absorbance spectrum.

Here, as an example, the number of levels (j: 1 to β) of the interference component sample (water) was set to 11 at intervals of 1° C. in the water temperature range of 30° C. to 40° C. That is, 11 samples up to 40° C. at which j=β=11 (for example, j=1 is 30° C., and j=2 is 31° C.) were measured. In addition, 90 measurement points (i: 1 to α) were set at intervals of 5 nm in the wavelength range of 800 nm to 1245 nm. That is, β samples were measured at 90 points up to 1245 nm at which i=α=90 (for example, i=1 has a wavelength of 800 nm, and i=2 has a wavelength of 805 nm).

In S04 shown in FIG. 3, a second sample signal (second sample vector Qⱼ) is formed based on the data of the absorbance spectrum obtained from the interference component sample (water). The second sample signal (second sample vector Qⱼ) corresponds to the measurement signal (measurement vector M) in FIG. 1, and is a measurement signal of the interference component having a known concentration. As shown in Equation (4), the second sample vector Qⱼ is expressed as a column vector of α rows by one column according to the measurement point i (1≦i≦α). β second sample vectors Qⱼ corresponding to the number of levels are formed.

Specifically, for example, an element Qᵢⱼ at the first row of the second sample vector Qⱼ of the j-th level is the absorbance at the wavelength of 800 nm of i=1 at the j-th water temperature. In addition, for example, an element Q_{αj} at the α-th row of the second sample vector Qⱼ is the absorbance at the wavelength of i=α (in this example, a wavelength of 1245 nm at α=90) at the j-th water temperature. Thus, αβ pieces of measurement data are expressed as β column vectors of α rows by one column. The second sample vector Qⱼ formed from the measured spectrum data is stored in the storage unit 50 as a second sample signal (interference component sample measurement signal data 531).

$$\vec{{Q}_{j}} = ( \begin{matrix}{Q}_{1 j} \\ ⋮ \\ ⋮ \\ ⋮ \\ {Q}_{α j}\end{matrix} ) \qquad ( 4 )$$

In step S05, the component analysis section 314 performs component analysis processing (multivariate analysis processing) on the second sample signal (second sample vector Qⱼ) acquired in step S04. As a result, an interference component feature quantity shown in step S06 is acquired. The multivariate analysis processing may utilize various analysis processes, such as an independent component analysis process or a main component analysis process. Among these, the independent component analysis process is preferable when detecting the signal of the high percentage component with high accuracy since the orthogonality of obtained interference vectors is strong and the independent component analysis process is excellent in error reduction.

By performing the independent component analysis process on the second sample signal (second sample vector Qⱼ) in step S05, an interference component feature quantity (interference unit vector Pₖ) that is a second sample feature signal (second feature signal) is obtained (step S06). The interference unit vector Pₖ (k is an integer of 1 to γ) is a column vector of α rows by one column, and γ is the number of independent components formed from the second sample vector Qⱼ. Here, since the number of independent components is 3, γ=3.

FIG. 4B shows the interference unit vector Pₖ acquired in step S06. In FIG. 4B, the horizontal axis indicates each element (i: 1 to α) of the interference unit vector Pₖ set at 90 points at intervals of 5 nm in a range of 800 nm to 1245 nm corresponding to the wavelength of light, and the vertical axis indicates the intensity of the absorbance spectrum. Since γ is 3 as described above, a first interference unit vector (first interference component feature quantity) P₁, a second interference unit vector (second interference component feature quantity) P₂, and a third interference unit vector (third interference component feature quantity) P₃ are extracted as three independent components contained in water that is an interference component. γ interference unit vectors Pₖ are stored as the interference component feature quantity data 541 in the storage unit 50.

The second sample vector Qⱼ is expressed as a linear sum of the interference unit vector Pₖ, as shown in Equation (5). In equation (5), μ_{kb} is a coefficient. For example, the second sample vector Q₁ of the first level when the water temperature is 30° C. (j=1) is expressed as a linear sum of the first interference unit vector P₁, the second interference unit vector P₂, and the third interference unit vector P₃ as shown in Equation (6).

$$\begin{matrix}\vec{{Q}_{j}} = {\sum }_{k = 1}^{γ} \vec{{P}_{k}} {μ}_{kj} {Q}_{ij} = {\sum }_{k = 1}^{γ} {P}_{ik} {μ}_{kj} & ( 5 ) \\ \vec{{Q}_{1}} = {\sum }_{k = 1}^{γ} \vec{{P}_{k}} {μ}_{k 1} = \vec{{P}_{1}} {μ}_{11} + \vec{{P}_{2}} {μ}_{21} + \vec{{P}_{3}} {μ}_{31} & ( 6 )\end{matrix}$$

As described above, the interference component feature quantity extraction process shown in FIG. 3 is ended.

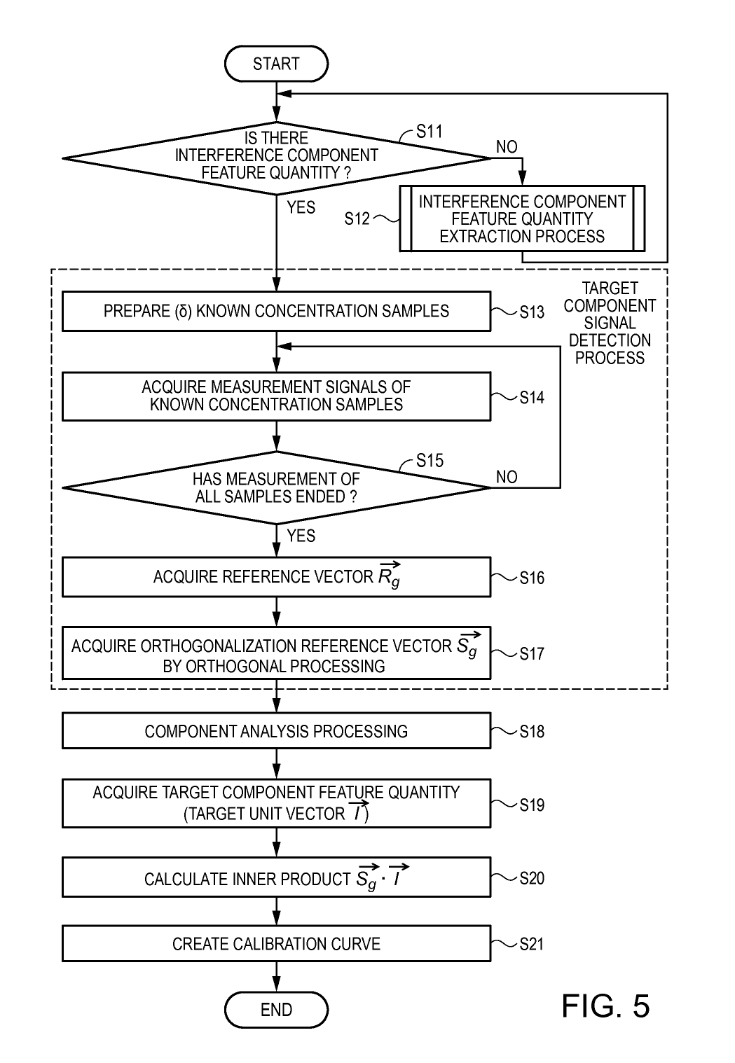

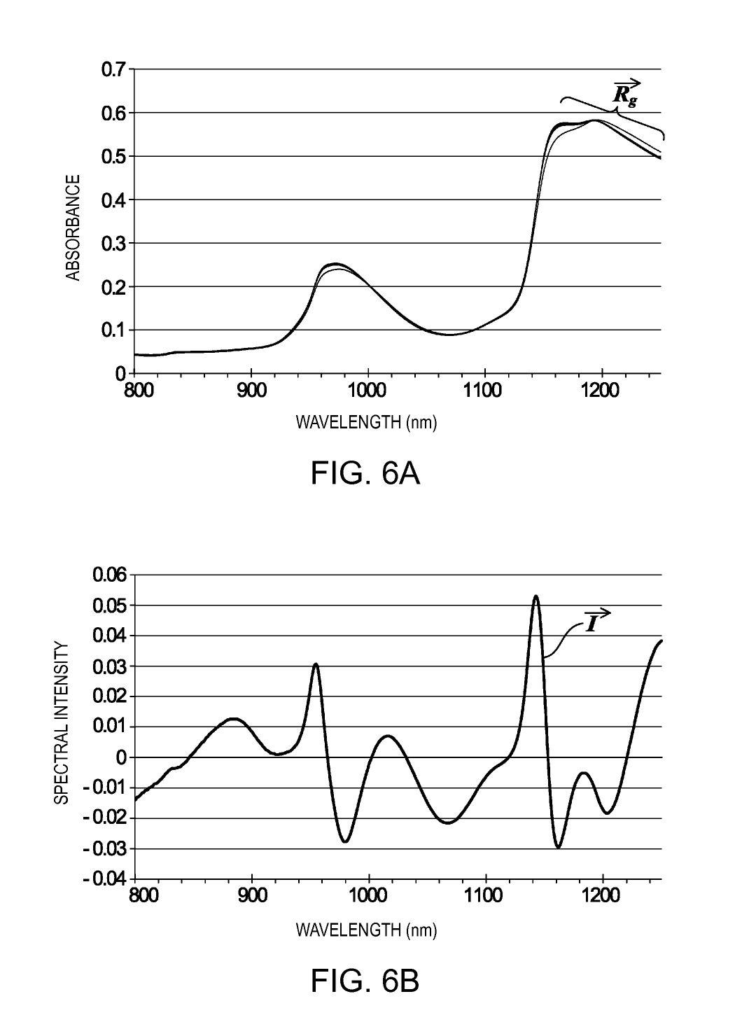

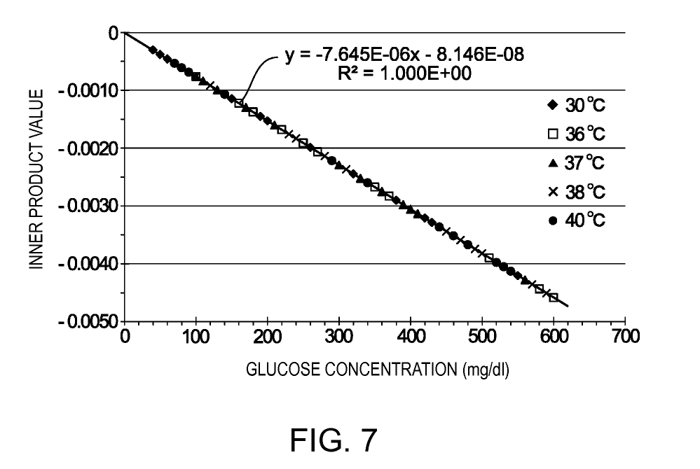

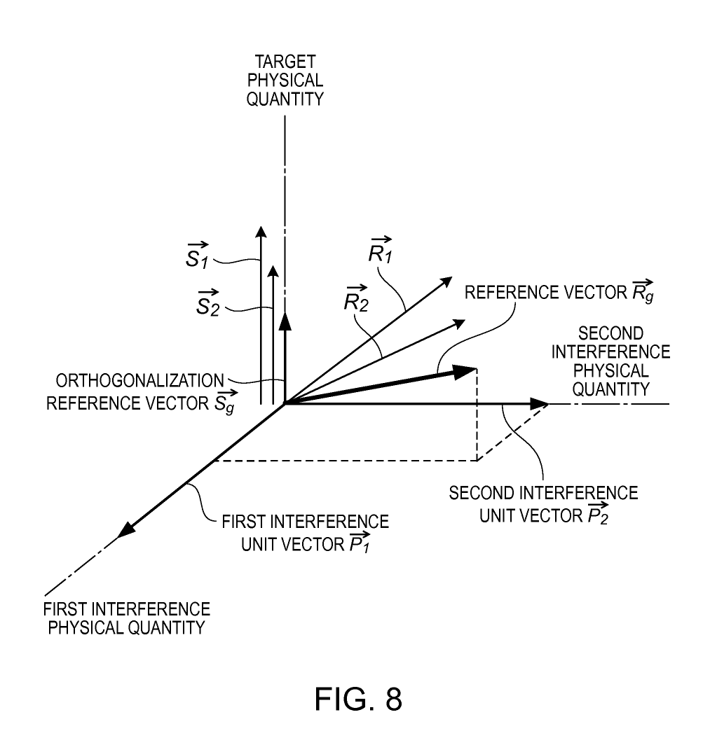

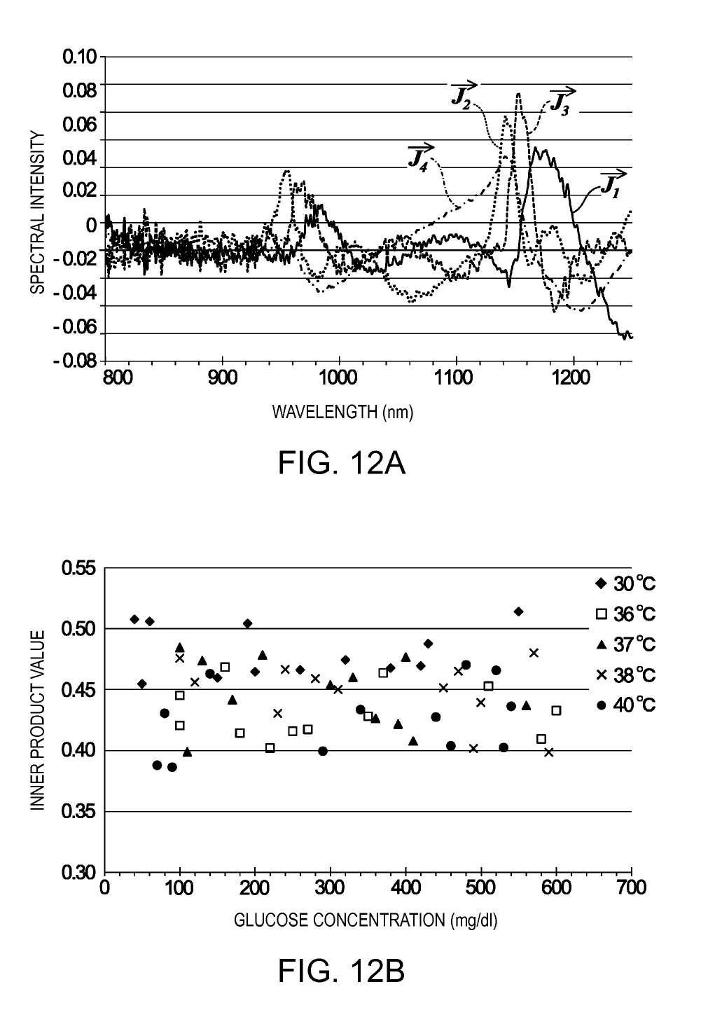

Next, the calibration curve creation process according to the first embodiment will be described. FIG. 5 is a flowchart showing the flow of the calibration curve creation process according to the first embodiment. FIGS. 6A and 6B are diagrams showing the data obtained by the calibration curve creation process according to the first embodiment. Specifically, FIG. 6A is a diagram showing an example of the absorbance spectrum obtained from the known concentration sample, and FIG. 6B is a diagram showing an example of the spectrum of the target component feature quantity. FIG. 7 is a diagram showing an example of the calibration curve created by the calibration curve creation process according to the first embodiment. FIG. 8 is a diagram for explaining the concept of an orthogonalization reference vector obtained by the projection operation according to the first embodiment. FIGS. 12A and 12B are diagrams showing the comparison data when performing quantification based on only the independent component analysis.

The calibration curve creation process is a process for creating a calibration curve for measuring the concentration of the target component. Therefore, before performing the concentration measurement process to be described later, it is necessary to create a calibration curve in advance. In addition, before performing the calibration curve creation process, the interference component feature quantity needs to be acquired in advance.

Therefore, first, when the interference component feature quantity is not stored as the interference component feature quantity data 541 in step S11 shown in FIG. 5 (step S11: NO), the interference component feature quantity extraction process of step S12 is performed. The interference component feature quantity extraction process shown in FIG. 3 corresponds to step S12. If the interference component feature quantity is acquired and stored as the interference component feature quantity data 541 in step S11 (step S11: YES), a target component signal detection process for detecting the signal of the target component is performed (steps S13 to S17).

In step S13, a reference sample is prepared in which the physical quantity of the target component relevant to the first signal is known. In the example of the present embodiment, a target component is glucose, and the physical quantity of the target component is the glucose concentration in the aqueous solution. Therefore, the reference sample is a known concentration sample having a known glucose concentration. Specifically, a plurality (δ; δ is an integer of 2 or more) of aqueous solutions having known and different concentrations of glucose that is a target component are prepared as known concentration samples (measurement targets). Since the spectrum data (or the composition ratio of feature quantities) of water that is an interference component changes with the temperature, it is preferable to prepare, as known concentration samples, not only the samples having different concentrations but also a plurality of samples obtained by changing the temperature as interference component samples.

Since the target component is a trace component having a concentration of 1% or less, the glucose concentration in any known concentration sample is assumed to be 1% or less. This is because the range of glucose concentration to be measured in the body is approximately 50 mg/dl to 600 mg/dl. The specific gravity of the blood is 1 g/cc that is the same as that of water, 1 dl (1 deciliter) is 100 g, and the glucose concentration is 1000 mg/dl or less. Accordingly, the glucose concentration is assumed to be 1% or less.

In step S14, the measurement signal of each of the δ aqueous glucose solutions having different concentrations, which are known concentration samples, is acquired. Here, similar to the case of the interference component sample, an absorbance spectrum is acquired as a measurement signal of the known concentration sample. The absorbance spectrum of the known concentration sample is acquired from the absorbance measuring device 6 through the measurement signal acquisition section 20, and is stored in the storage unit 50 as the known concentration sample measurement signal data 532. This is repeated until the measurement of 8 known concentration samples ends (step S15: NO to step S14).

When the measurement for all (8) known concentration samples ends (step S15: YES), data of the absorbance spectrum of each aqueous glucose solution that is a known concentration sample is obtained as a result. FIG. 6A shows the absorbance spectrum obtained from the known concentration sample (aqueous glucose solution). In FIG. 6A, the horizontal axis indicates a measurement point (i: 1 to α) corresponding to the wavelength of light, and the vertical axis indicates the absorbance.

Here, the number of levels δ of the aqueous glucose solution was set to 28 at intervals of 25 mg/dl in a range of 25 mg/dl to 700 mg/dl. That is, 28 samples up to 700 mg/dl at which g=δ=28 (for example, g=1 was a concentration of 25 mg/dl, and g=2 was a concentration of 50 mg/dl) were measured. In FIG. 6A, the absorbance spectra of the 28 aqueous glucose solutions having different concentrations are drawn so as to overlap each other. The measurement point (i: 1 to α) is set at 90 points at intervals of 5 nm in a range of 800 nm to 1245 nm.

In step S16 shown in FIG. 5, a reference vector R_{g} (g: 1 to δ) of the target component is acquired based on the data of the absorbance spectrum acquired from the known concentration sample (aqueous glucose solution). The reference vector R_{g} is obtained for each of the δ (=28) known concentration samples. As shown in Equation (7), the reference vector R_{g} is expressed as δ column vectors of α rows by one column according to the measurement point i (1≦i≦α) and the number of level g (1≦g≦δ). The acquired reference vector R_{g} is stored as the known concentration sample measurement signal data 532 in the storage unit 50 shown in FIG. 2.

$$\vec{{R}_{g}} = ( \begin{matrix}{R}_{1 g} \\ ⋮ \\ ⋮ \\ ⋮ \\ {R}_{α g}\end{matrix} ) \qquad ( 7 )$$

In step S17, orthogonal processing (orthogonal operation) for making the measurement signal (that is, the reference vector R_{g}) of the known concentration sample orthogonal to the signal of water, which is an interference component, is performed. In the first embodiment, a projection operation is used as the orthogonal operation. As shown in FIG. 8, a vector obtained by making the measurement signal (reference vector R_{g}) of the known concentration sample orthogonal to all of the γ interference unit vectors is set as an orthogonalization reference vector S_{g} of the target component, and the magnitude of the orthogonalization reference vector S_{g} (absolute value of the orthogonalization reference vector S_{g}) corresponds to the concentration. In the previous example, the number of interference unit vectors Pₖ is γ=3. In FIG. 8, however, in order to explain only the concept easily, only two of an interference unit vector P₁ and an interference unit vector P₂ are drawn as the interference unit vector Pₖ.

In the first embodiment, the orthogonalization reference vector S_{g} of the target component is calculated by performing a projection operation for projecting the measurement signal (reference vector R_{g}) of the known concentration sample to the orthogonal subspace extended by the second sample feature signal (second feature signal, interference unit vector Pₖ). The orthogonalization reference vector S_{g} of the target component is calculated by Equation (8).

\vec{S_{g}}=(E−P·P⁺)\vec{R_{g}} (8)

In Equation (8), E is a unit matrix of α rows by α columns, and is expressed by Equation (9). δᵢⱼ is a delta function.

$$E = ( \begin{matrix}1 & & & & \\ & 1 & & 0 & \\ & & ⋱ & & \\ & 0 & & ⋱ & \\ & & & & 1\end{matrix} ) = {δ}_{ij} = \{ \begin{matrix}i … i = j \\ 0 … i \neq j\end{matrix} \qquad ( 9 )$$

In Equation (8), P is an interference matrix of α rows by γ columns, and is a space extended by the γ interference unit vectors Pₖ as expressed by Equation (10).

$$P = ( \vec{{P}_{1}} … \vec{{P}_{γ}} ) = ( \begin{matrix}{P}_{11} & {P}_{12} & … & … & {P}_{1 γ} \\ ⋮ & ⋮ & & & ⋮ \\ ⋮ & ⋮ & & & ⋮ \\ ⋮ & ⋮ & & & ⋮ \\ {P}_{α1} & {P}_{α2} & … & … & {P}_{αγ}\end{matrix} ) \qquad ( 10 )$$

In Equation (8), P⁺ is a pseudo-inverse matrix of the interference matrix P, and is calculated by Equation (11).

P+(P^{T}p)⁻¹P^{T} (11)

In Equation (11), P⁺ is a transposed matrix of the interference matrix P, and is calculated by Equation (12). The transposed matrix P⁺ is a matrix of γ rows by α columns.

$${P}^{T} = ( \begin{matrix}{P}_{11} & … & … & … & {P}_{α1} \\ {P}_{12} & … & … & … & {P}_{α2} \\ ⋮ & & & & ⋮ \\ ⋮ & & & & ⋮ \\ {P}_{1 γ} & … & … & … & {P}_{αγ}\end{matrix} ) \qquad ( 12 )$$

By projecting the reference vector R_{g} to the orthogonal subspace extended by the second sample feature signal (second feature signal, interference unit vector Pₖ) by performing the projection operation shown in Equation (8), the orthogonalization reference vector S_{g} is obtained. The orthogonalization reference vector S_{g} is obtained for each of the δ (=28) known concentration samples. Since the orthogonalization reference vector S_{g} is orthogonal to the interference unit vector Pₖ, interference components are rarely contained.

As shown in Equation (13), the orthogonalization reference vector S_{g} is expressed as δ column vectors of α rows by one column according to the measurement point i (1≦i≦α) and the number of level g (1≦g≦δ). The acquired orthogonalization reference vector S_{g} is stored as the known concentration sample measurement signal data 532 in the storage unit 50.

$$\vec{{S}_{g}} = ( \begin{matrix}{S}_{1 g} \\ ⋮ \\ ⋮ \\ ⋮ \\ {S}_{α g}\end{matrix} ) \qquad ( 13 )$$

The target component signal detection process (steps S13 to S17) is performed according to the target component signal detection program 514, which is a subroutine program of the calibration curve creation program 510, by the first target component signal detecting section 316 shown in FIG. 2.

In step S18 shown in FIG. 5, the component analysis section 314 shown in FIG. 2 performs component analysis processing (multivariate analysis processing) on the orthogonalization reference vector S_{g} of the target component acquired by the orthogonal processing (projection operation). As the multivariate analysis processing, it is possible to use various analysis processes, such as an independent component analysis process or a main component analysis process. In the present embodiment, the independent component analysis process is performed. By performing the component analysis process on the orthogonalization reference vector S_{g} in step S18, a target component feature quantity (target unit vector I that is a unit signal of the first signal) is obtained (step S19).

The target unit vector I is orthogonal to the space where all of the interference unit vectors Pₖ extend (e.g., in FIG. 1, a plane defined by the first interference unit vector P₁ and the second interference unit vector P₂) even if the respective interference unit vectors Pₖ are not orthogonal to each other. Since there is one target component (glucose), one target component feature quantity is extracted by the component analysis processing. As shown in Equation (14), the target unit vector I is expressed as a column vector of α rows by one column corresponding to the measurement point i (1≦i≦α).

$$\vec{I} = ( \begin{matrix}{I}_{1} \\ ⋮ \\ ⋮ \\ ⋮ \\ {I}_{α }\end{matrix} ) \qquad ( 14 )$$

FIG. 6B shows an example of the spectrum of the target component feature quantity (target unit vector I) obtained in step S19. In FIG. 6B, the horizontal axis indicates a measurement point (i: 1 to α) corresponding to the wavelength of light, and the vertical axis indicates a spectral intensity. The acquired target unit vector I is stored as the target component feature quantity data 543 in the storage unit 50 shown in FIG. 2.

In step S20 shown in FIG. 5, the calibration curve creating section 310 calculates an inner product between the orthogonalization reference vector S_{g} and the target unit vector I as shown in Equation (15). Although the orthogonalization reference vector S_{g} is expressed as a column vector of α rows by one column as shown in Equation (13), an inner product between a row vector of one row by α columns that is the transposed matrix and the target unit vector I, which is expressed as a column vector of α rows by one column as shown in Equation (14), is calculated. By this inner product calculation, the magnitude of the orthogonalization reference vector S_{g} is determined as a scalar quantity.

$$\vec{{S}_{g}} \cdot \vec{I} = ( {S}_{1 g} … {S}_{αg} ) ( \begin{matrix}{I}_{1} \\ ⋮ \\ ⋮ \\ ⋮ \\ {I}_{α }\end{matrix} ) = {\sum }_{i = 1}^{α} {S}_{ig} {I}_{i} \qquad ( 15 )$$

By performing calculation as in Equation (15) for each level (g is an integer of 1 to δ; in this example, δ=28) corresponding to the concentration of aqueous glucose solution, the inner product value of each level is obtained as shown in Table 1. For example, the inner product value in the case of g=1 in which the concentration of the known concentration sample (aqueous glucose solution) is 25 mg/dl is calculated as in Equation (16).

\vec{S}·\vec{I}=S₁₁I₁+S₂₁I₂+ . . . S_{α1}I_{α} (16)

TABLE 1

| Level | Glucose concentration | Inner product value |
|---|---|---|
| g = 1 | 25 mg/dl | \vec{S₁} · \vec{I} |
| g = 2 | 50 mg/dl | \vec{S₂} · \vec{I} |
| . | . | . |
| . | . | . |
| . | . | . |
| g = δ = 28 | 700 mg/dl | \vec{S₂₈} · \vec{I} |

In step S21 shown in FIG. 5, the calibration curve creating section 310 creates a calibration curve showing the relationship between the physical quantity (in this example, known glucose concentration) of the target component and the inner product value obtained in step S20. The calibration curve created in step S21 is stored as the calibration curve data 545 in the storage unit 50. FIG. 7 shows an example of the calibration curve created in step S21.

In FIG. 7, the horizontal axis indicates the glucose concentration in Table 1, and the vertical axis indicates the inner product value in Table 1. Each point indicated by diamonds, squares, triangles, or circles shows the obtained calibration curve data, and the approximate straight line for the calibration curve data is also drawn. The approximate straight line is obtained by the least square method, and the equation of the obtained approximate straight line and a contribution ratio R² are described in FIG. 7. The contribution ratio R² is the square of a correlation coefficient R.

As shown in FIG. 7, the calibration curve data is on the approximate straight line, and the intercept of the equation of the approximate straight line passes through the origin in the error range. Accordingly, the contribution ratio R² becomes 1. Thus, it can be seen that the calibration curve data obtained in the present embodiment shows the very strong positive correlation between the concentration and the inner product value regardless of the temperature of the known concentration sample. This tells that the method of calculating the physical quantity of the target component according to the present embodiment is very accurate. Accordingly, the calibration curve obtained in step S21 is also very accurate. By using the calibration curve of the present embodiment, it is possible to calculate the target physical quantity with high accuracy even for a measurement target containing a target component with an unknown physical quantity.

As a comparative example, FIG. 12A shows a result when quantifying the absorbance spectrum of a known concentration sample using a method of independent component analysis. In the example shown in FIG. 12A, four components of J₁ to J₄ are extracted, and the spectrum of each component is calculated. Among J₁ to J₄, the spectrum of the component J₂, which shows a waveform close to a glucose that is a target component, is assumed to be target component feature quantity data. FIG. 12B is a diagram plotted by calculating the inner product value between the component J₂ and the reference vector R_{g} in order to create a calibration curve. As shown in FIG. 12B, in the case of quantification using a method of independent component analysis, a straight line passing through the plot cannot be drawn. Therefore, it is not possible to create a calibration curve. This indicates that a trace of target component cannot be independently separated in the independent component analysis.

As described in the present embodiment, the method of calculating the orthogonalization reference vector S_{g} of the target component improves the quantification of the trace component. Specifically, the quantification of the trace component is achieved by performing calculations to adjust the reference vector R_{g} such that the reference vector R_{g} is orthogonal to the second feature signal (orthogonal subspace extended by all of the interference unit vectors Pₖ), calculating the target unit vector I from the orthogonalization reference vector S_{g}, and calculating an inner product between the reference vector R_{g} and the target unit vector I.

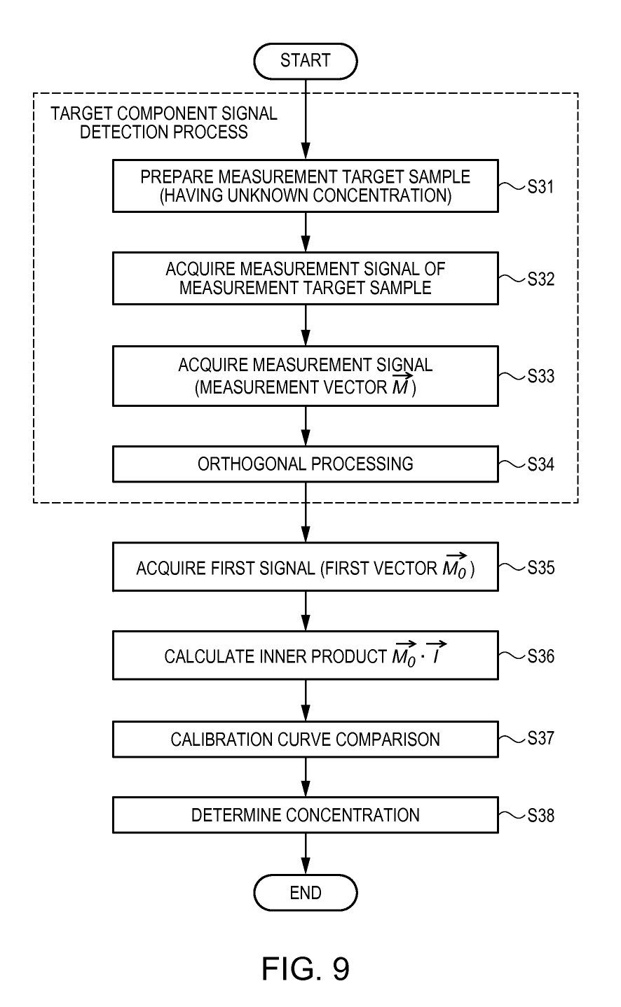

Next, the concentration measurement process according to the first embodiment will be described. FIG. 9 is a flowchart showing the flow of the concentration measurement process according to the first embodiment. The concentration measurement process is a process for measuring the concentration of the target component, which is a trace component, from the measurement target sample having an unknown concentration. In the concentration measurement process, the calibration curve for measuring the concentration of the target component is referred to. Therefore, as stated above, before performing the concentration measurement process, the calibration curve is created in advance by performing the above-described calibration curve creation process.

Steps S31 to S34 shown in FIG. 9 are steps of performing a target component signal detection process for detecting the signal of the target component from the measurement target sample having an unknown concentration. First, in step S31, a measurement target sample having an unknown concentration is prepared. In the present embodiment, an aqueous solution containing glucose as a target component whose concentration is unknown is prepared as a measurement target sample.

In step S32, a measurement signal of the measurement target sample (aqueous glucose solution having an unknown concentration) is acquired. Similar to the case of the known concentration sample, the absorbance spectrum of the measurement target sample is acquired as a measurement signal. Glucose that is a target component and water that is an interference component are contained in the measurement target sample. Accordingly, the measurement signal includes a signal (first signal) of the target component, and a signal (second signal) of the interference component. The absorbance spectrum of the measurement target sample is acquired from the absorbance measuring device 6 through the measurement signal acquisition section 20, and is stored in the storage unit 50 as the concentration measurement target measurement signal data 533.

In step S33, the second target component signal detecting section 322 acquires the measurement vector M based on the data of the absorbance spectrum acquired from the measurement target sample (aqueous glucose solution having an unknown concentration). As shown in Equation (17), the measurement vector M is expressed as a column vector of α rows by one column according to the measurement point i (1≦i≦α).

$$\vec{M} = ( \begin{matrix}{M}_{1} \\ ⋮ \\ ⋮ \\ ⋮ \\ {M}_{α }\end{matrix} ) \qquad ( 17 )$$

In step S34, the second target component signal detecting section 322 performs orthogonal processing (orthogonal operation) for making the measurement signal of the measurement target sample (aqueous glucose solution having an unknown concentration) orthogonal to the signal of water, which is an interference component. Similar to step S17 in the calibration curve creation process of FIG. 5, a projection operation for making the measurement signal of the measurement target sample orthogonal to the second feature signal (orthogonal subspace extended by all of the interference unit vectors Pₖ) is used as the orthogonal operation.

As shown in FIG. 1, the measurement signal (measurement vector M) of the measurement target sample is expressed as a linear sum of the first signal (first vector M₀) of the target component and γ interference component feature quantities (γ interference unit vectors; in the example shown in FIG. 1, the first interference unit vector P₁ and the second interference unit vector P₂) of interference components.

Therefore, the first signal (first vector M₀) of the target component is calculated by performing a projection operation for projecting the measurement signal (measurement vector M) of the measurement target sample upon the orthogonal subspace extended by the second signal (interference unit vector Pₖ). The first signal (first vector M₀) of the target component is calculated by Equation (18). In Equation (18), E and P are expressed by Equations (9) and (10) described above, respectively, and P⁺ is expressed by Equations (11) and (12) described above.

\vec{M₀}=(E−P·P⁺)\vec{M} (18)

Thus, the first signal (first vector M₀) of the target component is acquired from the measurement signal (measurement vector M) of the measurement target sample (step S35). The first vector M₀ is orthogonal to the space where all of γ interference unit vectors extend (e.g., in FIG. 1, a plane defined by the first interference unit vector P₁ and the second interference unit vector P₂) even if the respective interference unit vectors Pₖ are not orthogonal to each other. In addition, the first signal (first vector M₀) of the target component calculated herein is spectrum data indicating the strength for each wavelength.

In step S36, as shown in Equation (19), the concentration measuring section 320 calculates an inner product between the first vector M₀ of the target component acquired in step S35 and the target unit vector I stored as the target component feature quantity data 543 in the storage unit 50. Through this inner product calculation, as shown in FIG. 1, the absolute value of the first vector M₀ is calculated as a scalar quantity m₀.

$${m}_{0} = \vec{{M}_{0}} \cdot \vec{I} = ( {M}_{01} … {M}_{0 α} ) ( \begin{matrix}{I}_{1} \\ ⋮ \\ ⋮ \\ ⋮ \\ {I}_{α }\end{matrix} ) = {\sum }_{i = 1}^{α} {M}_{0 i} {I}_{i} = {M}_{01} {I}_{1} + {M}_{02} {I}_{2} + … + {M}_{0 α} {I}_{α} \qquad ( 19 )$$

In step S37, the concentration measuring section 320 determines the glucose concentration of the measurement target sample by comparing the concentration corresponding to the inner product value m₀ acquired in step S36 with the calibration curve data 545 (calibration curve of in FIG. 7) stored in the storage unit (step S38). More specifically, in the calibration curve shown in FIG. 7, a value of the horizontal axis when the inner product value calculated in step S36 is assumed to be the value of the vertical axis is glucose concentration to be calculated. Therefore, it is possible to measure the concentration of glucose that is a target component contained in the measurement target sample.

As described above, according to the signal detection device 1, the signal detection method, the calibration curve creation method, and the quantification method of the first embodiment, the first signal (first vector M₀) relevant to glucose that is a target component contained in the measurement target can be accurately detected from the measurement signal (measurement vector M) obtained by measuring the measurement target. In addition, it is possible to create a calibration curve correctly using the detection of the target component feature quantity (target unit vector I). Therefore, it is possible to correctly measure the concentration of the target component contained as a trace component in the measurement target.

### Second Embodiment

Next, a second embodiment will be described. In the second embodiment, the configuration of the signal detection device 1 is the same as that in the first embodiment, and the signal detection method, the calibration curve creation method, and the quantification method are almost the same as those in the first embodiment except that an orthogonalization method of Gram-Schmidt is used as an orthogonal operation in the calibration curve creation process and the concentration measurement process. Here, the method of orthogonal operation according to the second embodiment will be described focusing on the differences from the first embodiment.

In the calibration curve creation process of the second embodiment, in the orthogonal processing of step S17 shown in FIG. 5, an orthogonalization method of Gram-Schmidt using the interference unit vector Pₖ is applied for the reference vector R_{g} of the known concentration sample, instead of performing a projection operation for projecting the reference vector R_{g} of the known concentration sample to the orthogonal subspace extended by the interference unit vector Pₖ.

In the second embodiment, an intermediate vector Wₖ is formed by sequentially orthogonalizing the interference component feature quantity (interference unit vector Pₖ shown in FIG. 4B) extracted by the interference component feature quantity extraction process. k (k=1 to γ) is the number of interference unit vectors as in the first embodiment. A first intermediate vector W₁ that is obtained from the first interference unit vector P₁ using the orthogonalization method of Gram-Schmidt is expressed by Equation (20).

\vec{W₁}=\vec{P₁} (20)

Then, a second intermediate vector W₂ corresponding to the second interference unit vector P₂ is made to be orthogonal to the first intermediate vector W₁, and a third intermediate vector W₃ corresponding to the third interference unit vector P₃ is made to be orthogonal to the first intermediate vector W₁ and the second intermediate vector W₂. In this manner, sequential orthogonalization is performed. Therefore, the respective intermediate vectors Wₖ are orthogonal to each other. An intermediate vector Wₜ (t=2 to γ) corresponding to the interference unit vector Pₜ is expressed by Equation (21).

$$\vec{{W}_{t}} = \vec{{P}_{t}} - {\sum }_{i = 1}^{t - 1} \frac{{\vec{{W}_{i}}}^{T} \cdot \vec{{P}_{t}}}{{\vec{{W}_{i}}}^{T} \cdot \vec{{W}_{i}}} \cdot \vec{{W}_{i}} , t = 2 … γ \qquad ( 21 )$$

Assuming that the number of independent components is three (γ=3), from Equation (21), the second intermediate vector W₂ is expressed by Equation (22), and the third intermediate vector W₃ is expressed by Equation (23).

$$\begin{matrix} \vec{{W}_{2}} = \vec{{P}_{2}} - \frac{{\vec{{W}_{1}}}^{T} \cdot \vec{{P}_{2}}}{{\vec{{W}_{1}}}^{T} \cdot \vec{{W}_{1}}} \cdot \vec{{W}_{1}} = \vec{{P}_{2}} - \frac{{\vec{{P}_{1}}}^{T} \cdot \vec{{P}_{2}}}{{\vec{{P}_{1}}}^{T} \cdot \vec{{P}_{1}}} \cdot \vec{{P}_{1}} & ( 22 ) \\ \vec{{W}_{3}} = \vec{{P}_{3}} - \frac{{\vec{{W}_{1}}}^{T} \cdot \vec{{P}_{3}}}{{\vec{{W}_{1}}}^{T} \cdot \vec{{W}_{1}}} \cdot \vec{{W}_{1}} - \frac{{\vec{{W}_{2}}}^{T} \cdot \vec{{P}_{3}}}{{\vec{{W}_{2}}}^{T} \cdot \vec{{W}_{2}}} \cdot \vec{{W}_{2}} = \vec{{P}_{3}} - \frac{{\vec{{P}_{1}}}^{T} \cdot \vec{{P}_{3}}}{{\vec{{P}_{1}}}^{T} \cdot \vec{{P}_{1}}} \cdot \vec{{P}_{1}} - \frac{{\vec{{W}_{2}}}^{T} \cdot \vec{{P}_{3}}}{{\vec{{W}_{2}}}^{T} \cdot \vec{{W}_{2}}} \cdot \vec{{W}_{2}} & ( 23 )\end{matrix}$$

In the orthogonalization method of Gram-Schmidt, the orthogonalization reference vector S_{g} obtained in step S17 shown in FIG. 5 is expressed by Equation (24). The orthogonalization reference vector S_{g} is orthogonal to each intermediate vector Wₖ. Therefore, the orthogonalization reference vector S_{g} is also orthogonal to the linear sum of the respective intermediate vectors Wₖ.

$$\vec{{S}_{g}} = \vec{{R}_{g}} - {\sum }_{i = 1}^{γ} \frac{{\vec{{W}_{i}}}^{T} \cdot \vec{{R}_{g}}}{{\vec{{W}_{i}}}^{T} \cdot \vec{{W}_{i}}} \cdot \vec{{W}_{i}} \qquad ( 24 )$$

Thereafter, as in the first embodiment, by performing the component analysis processing (multivariate analysis processing) on the orthogonalization reference vector S_{g} in step S18 shown in FIG. 5, a target component feature quantity (target unit vector I) is obtained (step S19). Even if the respective interference unit vectors Pₖ are not orthogonal to each other, the respective intermediate vectors Wₖ are orthogonal to each other. Accordingly, the target unit vector I is orthogonal to the space where all of the interference unit vectors Pₖ extend (that is, space where all of the interference vectors Wₖ extend).

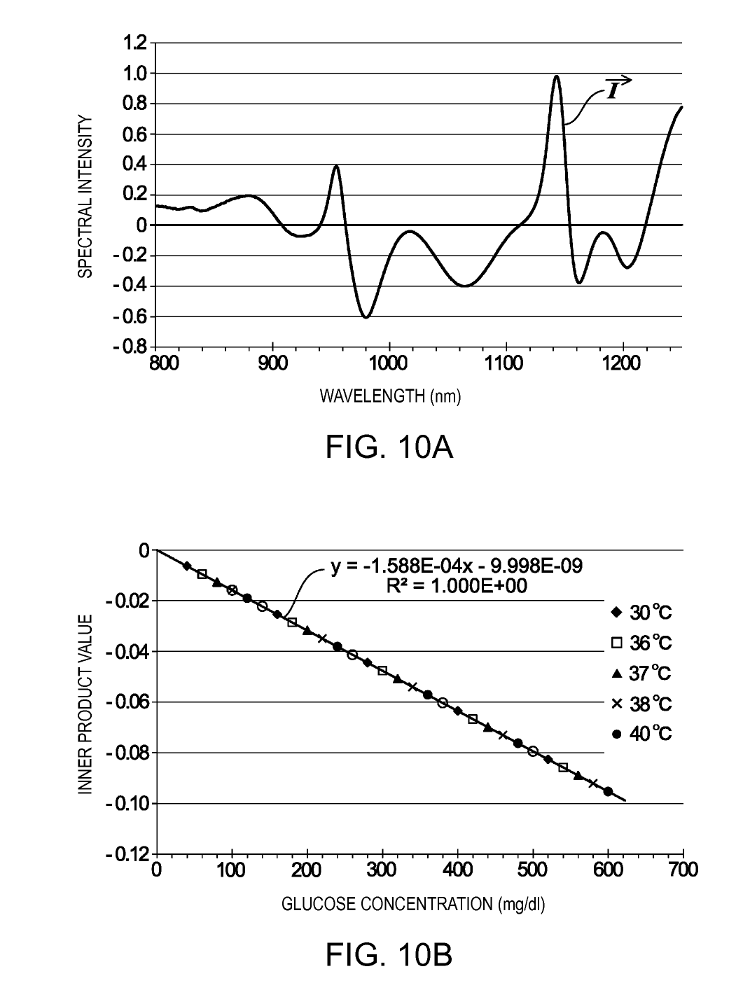

FIGS. 10A and 10B are diagrams showing the data obtained by the calibration curve creation process according to the second embodiment. FIG. 10A shows the spectrum of the target component feature quantity (target unit vector I) obtained in step S19 of the second embodiment. In FIG. 10A, the horizontal axis indicates a measurement point (i: 1 to α) corresponding to the wavelength of light, and the vertical axis indicates the spectral intensity. As shown in FIG. 10A, also in the second embodiment, the same spectrum as the spectrum obtained in the first embodiment shown in FIG. 6B is obtained.

In step S20 shown in FIG. 5, an inner product between the orthogonalization reference vector S_{g} and the target unit vector I is calculated. Then, a calibration curve is created based on the inner product value obtained by the inner product calculation (step S21).

FIG. 10B shows the calibration curve created in step S21 of the second embodiment. In FIG. 10B, the equation of the approximate straight line obtained by the least square method for the calibration curve data and the contribution ratio R² are described. As shown in FIG. 10B, the calibration curve data is on the approximate straight line, and the intercept of the equation of the approximate straight line passes through the origin in the error range. Accordingly, the contribution ratio R² becomes 1. Therefore, also in the second embodiment, it can be seen that the calibration curve is obtained with high accuracy as in the first embodiment.

Next, in the concentration measurement process of the second embodiment, in the orthogonal processing of step S34 shown in FIG. 9, an orthogonalization method of Gram-Schmidt using the interference unit vector Pₖ is applied for the measurement vector M of the measurement target sample, instead of performing a projection operation for projecting the measurement vector M upon the orthogonal subspace extended by the interference unit vector Pₖ.

The intermediate vector Wₖ is calculated from Equations (20) to (23) described above. The first vector M₀ obtained by the orthogonalization method of Gram-Schmidt in step S34 is expressed by Equation (25). The first vector M₀ is orthogonal to each intermediate vector Wₖ. In addition, even if the respective interference unit vectors Pₖ are not orthogonal to each other, the first vector M₀ is orthogonal to the space where all of the γ interference unit vectors Pₖ extend.

$$\vec{{M}_{0}} = \vec{M} - {\sum }_{i = 1}^{γ} \frac{{\vec{{W}_{i}}}^{T} \cdot \vec{M}}{{\vec{{W}_{i}}}^{T} \cdot \vec{{W}_{i}}} \cdot \vec{{W}_{i}} \qquad ( 25 )$$

As an example, the first vector M₀ when the number of interference unit vectors Pₖ is three (7=3) is described in Equation (26).

$$\vec{{M}_{0}} = \vec{M} - \frac{{\vec{{W}_{1}}}^{T} \cdot \vec{M}}{{\vec{{W}_{1}}}^{T} \cdot \vec{{W}_{1}}} \cdot \vec{{W}_{1}} - \frac{{\vec{{W}_{2}}}^{T} \cdot \vec{M}}{{\vec{{W}_{2}}}^{T} \cdot \vec{{W}_{2}}} \cdot \vec{{W}_{2}} - \frac{{\vec{{W}_{3}}}^{T} \cdot \vec{M}}{{\vec{{W}_{3}}}^{T} \cdot \vec{{W}_{3}}} \cdot \vec{{W}_{3}} \qquad ( 26 )$$

Thereafter, as in the first embodiment, by performing steps S35 to S38 shown in FIG. 9, it is possible to measure the concentration of glucose that is a target component contained in the measurement target sample.

As described above, also in the second embodiment, the first signal (first vector M₀) relevant to glucose that is a target component contained in the measurement target can be accurately detected from the measurement signal (measurement vector M) obtained by measuring the measurement target. In addition, it is possible to create a calibration curve correctly using the detection of the target component feature quantity (target unit vector I). Therefore, it is possible to correctly measure the concentration of the target component contained as a trace component in the measurement target.

### Third Embodiment

Next, a third embodiment will be described. In the third embodiment, the configuration of the signal detection device, the signal detection method, the calibration curve creation method, and the quantification method are the same as those in the first embodiment or the second embodiment, but the applications are different.

That is, in the third embodiment, the human body fluid is used as a measurement target, and the concentration of a specific trace component in the body fluid is measured. As the body fluid, it is possible to use blood, lymph, tissue fluid, sweat, and urine, for example. As a target component (trace component) whose concentration is to be measured, glucose, cholesterol, or triglyceride can be used when the body fluid is blood, and uric acid or sugar can be used when the body fluid is urine.

Also in the third embodiment, an interference component contained in the measurement target can be water. Accordingly, the interference component sample is water. In addition, the known concentration samples need to be a plurality of samples containing target components whose concentrations are to be measured and which have different concentrations. Therefore, for example, body fluids collected at various times, places, and conditions in daily life are used as known concentration samples.

Since water that is a high percentage component contained in the body fluid has a characteristic that spectrum data (or the composition ratio of feature quantities) changes with temperature, it is preferable to further prepare a plurality of known concentration samples by changing the temperature of the sampled body fluids. For example, if the body fluid that is a measurement target is blood and the target component is blood sugar, blood before and after a meal, blood before and after an exercise, or blood before and after going to bed can be collected, and the blood sugar level can be measured using a separate measuring device to prepare a known concentration sample.

In the third embodiment, the body fluid that has been actually collected is used as a known concentration sample. However, it is also possible to create a sample by simulating the body fluid and use the sample.

The embodiments described above are for illustrative purposes, and modifications and applications may be arbitrarily made within the scope of the present disclosure. As modification examples, the following examples can be considered.

### Modification Example 1

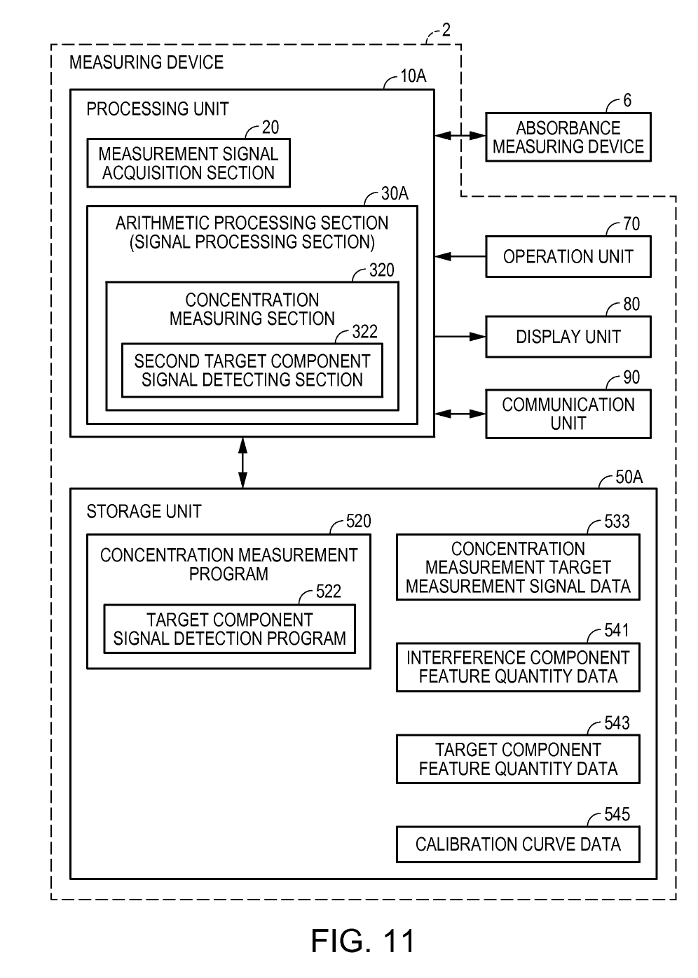

In the embodiments described above, the signal detection device 1 is configured to have the signal detection device, the calibration curve creation device, and the measuring device. However, the present disclosure is not limited to the embodiments described above. For example, if the interference component feature quantity extraction process and the calibration curve creation process are performed separately, the operation of the signal detection device and the calibration curve creation device can be separated from the signal detection device 1 of the embodiments described above. Therefore, it is possible to provide a measuring device specialized for the concentration measurement process. FIG. 11 is a block diagram illustrating the configuration of a measuring device according to a modification example 1.

As shown in FIG. 11, a measuring device 2 includes a processing unit 10A, a storage unit 50A, an operation unit 70, a display unit 80, and a communication unit 90. The processing unit 10A includes a measurement signal acquisition section 20 and an arithmetic processing section 30A. The arithmetic processing section 30A includes a concentration measuring section 320, and does not include the calibration curve creating section 310, which is provided with the arithmetic processing section 30 of FIG. 2. The storage unit 50A stores the concentration measurement program 520, but does not store the calibration curve creation program 510. The storage unit 50A stores the interference component feature quantity data 541 (interference unit vector Pₖ), the target component feature quantity data 543 (target unit vector I), and the calibration curve data 545 that are acquired in advance. The storage unit 50A also stores the concentration measurement target measurement signal data 533 that is calculated when executing the concentration measurement process.

The measurement signal acquisition section 20 executes steps S32 and S33 of FIG. 9. The second target component signal detecting section 322 executes steps S34 and S35 of FIG. 9 using the interference component feature quantity data 541. Using the target component feature quantity data 543, the concentration measuring section 320 calculates the inner product at step 36 and, using the calibration curve data 545, measures the concentration at steps S37 and S38 of FIG. 9. The measured concentration is displayed on the display unit 80, or is transmitted to other electronic devices (for example, a smartphone or a large-scale storage device, such as a server) through the communication unit 90.

According to the configuration of the measuring device 2 shown in the modification example 1, when a measurement target and a target component and an interference component contained in the measurement target can be specified, it is possible to provide a device capable of measuring the concentration of the target component contained in the measurement target at a lower cost.

### Modification Example 2

Application of the present disclosure should not be limited to the embodiments described above. For example, the present disclosure can also be applied to an embodiment for measuring the concentration or amount of impurities that are trace components that may be contained in the ingredients of drug, an embodiment for detecting a frequency signal with low amplitude that may be included in the radio wave, an embodiment for detecting the magneto-cardiogram of a person that is a trace component under the environment in which there is a magnetic interference component, such as geomagnetism, an embodiment for detecting a small abnormal amplitude signal embedded in the pulse wave signal of blood, and the like. In addition, when detecting a defective pixel using a test device for a display, the present disclosure can also be applied to a method of detecting the signal of a defective pixel from the display (interference component) of the entire screen. In addition, the present disclosure can also be applied to an algorithm for detecting the fingerprint of a specific person among many fingerprints.

### Modification Example 3

In the embodiments described above, as an example of the orthogonal operation in the orthogonal processing of steps S17 and S34 of FIGS. 5 and 9, respectively, the projection operation has been mentioned in the first embodiment, and the orthogonalization method of Gram-Schmidt has been mentioned in the second embodiment. However, it is also possible to realize the orthogonal operation using other orthogonalization methods, such as a symmetric orthogonalization method based on a repetition method.

### Modification Example 4

In the embodiments described above, the independent component analysis has been used for the component analysis process of steps S05 and S18. However, the component analysis process is not limited to the independent component analysis as long as it is a multivariate analysis. For example, principal component analysis or the Fourier transform may be applied. As described in detail in the first embodiment, since a target component is orthogonal to all interference components, each of the interference vectors does not need to be orthogonal to each other. However, since the interference vectors acquired in the independent component analysis are strongly orthogonal to each other, the independent component analysis can be used to reduce error.

The entire disclosure of Japanese Patent Application Nos. 2014-206460 filed on Oct. 7, 2014; 2014-210486 filed Oct. 15, 2014; and 2015-098825 filed May 14, 2015 are incorporated by reference herein.

What is claimed is:

1. A signal detection method comprising:
acquiring a measurement signal, wherein the measurement signal includes a first signal and a second signal different from the first signal; and
performing an orthogonal operation for adjusting the measurement signal such that the measurement signal is orthogonal to the second signal.

2. The signal detection method according to claim 1 wherein:
the orthogonal operation utilizes a second feature signal obtained by performing a multivariate analysis process of a second sample signal, and
the second sample signal is obtained by measuring a sample that contains a component relevant to the second signal and does not contain a component relevant to the first signal.

3. The signal detection method according to claim 2 wherein the multivariate analysis process is an independent component analysis.

4. The signal detection method according to claim 2 wherein the orthogonal operation includes a projection operation that projects the measurement signal to a first space orthogonal to a second space defined by the second feature signal.

5. The signal detection method according to claim 4 wherein, with the measurement signal provided as a measurement vector M, the first signal provided as a first vector M₀, the second feature signal provided as γ interference unit vectors Pₖ, the space extended by the second feature signal provided as a matrix P including the interference unit vectors Pₖ, a pseudo-inverse matrix of the matrix P provided as P⁺, and a unit matrix provided as E, the projection operation is expressed by the following equation:
\vec{M₀}=(E−P·P⁺)\vec{M}.

6. The signal detection method according to claim 2 wherein the orthogonal operation includes an orthogonalization method of Gram-Schmidt that uses the second feature signal.

7. The signal detection method according to claim 6 wherein, with the measurement signal provided as a measurement vector M, the first signal provided as a first vector M₀, the second feature signal provided as γ interference unit vectors Pₖ, γ intermediate vectors provided as Wₖ, and transposed vectors of the intermediate vectors Wₖ provided as Wₖ^{T}, the orthogonalization method of Gram-Schmidt is provided by the following equations with a first intermediate vector W₁ as a first interference unit vector P₁:
$$\vec{{W}_{t}} = \vec{{P}_{t}} - {\sum }_{i = 1}^{t - 1} \frac{{\vec{{W}_{i}}}^{T} \cdot \vec{{P}_{t}}}{{\vec{{W}_{i}}}^{T} \cdot \vec{{W}_{i}}} \cdot {W}_{i} , t = 2 … γ$$
$$\vec{{M}_{0}} = \vec{M} - {\sum }_{i = 1}^{γ} \frac{{\vec{{W}_{i}}}^{T} \cdot \vec{M}}{{\vec{{W}_{i}}}^{T} \cdot \vec{{W}_{i}}} \cdot {W}_{i} .$$

8. The signal detection method according to claim 1 wherein a percentage of the first signal in the measurement signal is equal to or less than 1%.

9. The signal detection method according to claim 1 wherein a percentage of the second signal in the measurement signal is equal to or greater than 3%.

10. The signal detection method according to claim 1 wherein the second signal includes spectrum data of water.

11. The signal detection method according to claim 10 wherein the spectrum data includes spectrum data at a plurality of different temperatures.

12. A calibration curve creation method comprising:
acquiring a measurement signal for a reference sample, wherein the measurement signal includes a first signal and a second signal different from the first signal, and the reference sample has a predetermined physical quantity relevant to the first signal;
performing an orthogonal operation for adjusting the measurement signal such that the measurement signal is orthogonal to the second signal;
determining the first signal based on the orthogonal operation of the measurement signal;
calculating an inner product value between the first signal and a unit signal of the first signal; and
generating a calibration curve, wherein the calibration curve associates a physical quantity relevant to the first signal with the inner product value.

13. A quantification method comprising:
acquiring a measurement signal, wherein the measurement signal includes a first signal and a second signal different from the first signal;
performing an orthogonal operation for adjusting the measurement signal such that the measurement signal is orthogonal to the second signal; and
calculating an inner product value between the first signal and a unit signal of the first signal, wherein the first signal is based on the orthogonal operation of the measurement signal.

14. The quantification method according to claim 13, further comprising:
quantifying a physical quantity with reference to the inner product value and a calibration curve.

15. The quantification method according to claim 14 further comprising:
generating the calibration curve, wherein the generation of the calibration curve further includes:
acquiring a measurement signal for a reference sample, wherein the measurement signal includes a first signal and a second signal different from the first signal, and the reference sample has a predetermined physical quantity relevant to the first signal;
performing an orthogonal operation for adjusting the measurement signal such that the measurement signal is orthogonal to the second signal;
determining the first signal based on the orthogonal operation of the measurement signal;
calculating an inner product value between the first signal and a unit signal of the first signal, wherein the calibration curve associates a respective physical quantity relevant to the first signal with a respective inner product value.

16. The quantification method according to claim 14 wherein the physical quantity is glucose concentration in blood.

17. A signal detection device comprising:
an acquisition unit that acquires a measurement signal, wherein the acquisition unit measures a measurement target containing a component relevant to a first signal and a component relevant to a second signal different from the first signal; and
an arithmetic processing unit that performs an orthogonal operation, wherein the orthogonal operation adjusts the measurement signal such that the measurement signal is orthogonal to the second signal.

18. A measuring device comprising:
an acquisition unit that acquires a measurement signal, wherein the acquisition unit measures a measurement target containing a component relevant to a first signal and a component relevant to a second signal different from the first signal; and
an arithmetic processing unit that performs an orthogonal operation for adjusting the measurement signal such that the measurement signal is orthogonal to the second signal, wherein the arithmetic processing unit quantifies a physical quantity using a result of the orthogonal operation.

19. A glucose concentration measuring device comprising the signal detection device according to claim 17.

20. A glucose concentration measuring device comprising the measuring device according to claim 18.
